(19) United States
(12) Patent Application Publication
(10) Pub. No.: US 2006/0172406 A1
(43) Pub. Date: Aug. 3, 2006

(54) Method and apparatus for production and refinement of microbial consortia and method of use of microbial consortia as therapeutic agent

(76) Inventors: Charles R. Pellegrino, Long Beach, NY (US); Lori Johnston, Regina (CA); Roy Cullimore, Regina (CA); Mark Newman, Port Jefferson Station, NY (US)

Correspondence Address: ADAMS & WILKS, 31st Floor, 50 Broadway, New York, NY, 10004, US

(21) Appl. No.: 10/812,436

(22) Filed: Mar. 30, 2004

(51) Int. Cl. C12N 1/20; C12M 3/00

(52) U.S. Cl. 435/252.4; 435/289.1

(57) ABSTRACT

A method of producing a rusticle consortia includes the steps of placing one or more samples obtained from a rusticle on a growth substrate, placing the growth substrate with the samples thereon into a tank filled with an aqueous solution, and controlling an environment of the aqueous solution to promote growth of the rusticle on the growth substrate. The growth substrate may comprise an anodically charged metal such as a mild steel plate that has been embrittled or unlike metals such as Fe and Cu. The aqueous solution is preferably water containing an oxygen and nutrients sufficient to promote growth of the rusticles. A charge may be applied to a location near the growth substrate to form a concentration of particles given off by the rusticle. In preferred embodiments, the grown rusticles are used as therapeutic agents such as anti-microbial, anti-biotic, or anti-cancer agents.

### BACKGROUND OF THE INVENTION

1. Field of the Invention

The present invention relates generally to the formation, testing and use of microbial consortia and, more particularly, to the production of a mass culture of a consortia for the purpose of generating a desired product, to methods for the optimization and refinement of the product, and to methods of use of the product as a therapeutic agent such as anti-microbial and anti-cancer agents.

2. Discussion of the Related Art

Over the last one hundred years or so, advances in the field of microbiology have largely been based upon the premise that single species of microorganisms are capable of producing efficiently useful products for humankind. These products have ranged from organic solvents and selected biochemicals, to antibiotics and plasmids. In all of these developments, there has been a preoccupation with the utilization of a single species, refinement using common agar plate technologies, and development of a single specific chemical compound. Heretofore, virtually no attention has been paid to the use of bacterial consortia to either generate a defined product or compete successfully with a nuisance or pathogenic microorganism or viral particle. Over the past century, the scientific community has generally overlooked the synergistic activities of species within consortia to achieve sophisticated products, conditions and effects.

Historically, for instance, microbiology has pursued a pathway developed by other natural sciences and, in particular, zoology, botany and chemistry. From the former two disciplines, the concept was adopted to separate and study microbial species as independent entities in a manner similar to that observed for species of plants and animals. From the latter discipline, the differentiation of microorganisms has been achieved at a molecular level. This combination of interest foci (at the species and molecular levels) has led to a lack of development in the understanding of microbial communities and the manner with which these consorms are able to collectively generate specific agents of interest.

The acceptance of standard microbiological practices involving agar cultural techniques and traditional broth techniques has limited scientific discoveries to those that are directly attributable to single-species events often commonly reproducible on agar media or traditional cultural technologies.

In view of the foregoing shortcomings of the prior art, it would be desirable to produce a mass culture of a consortia as a vehicle for generating a therapeutic or otherwise useful agent.

At its most fundamental level, the nature of most microbial activity is the common practice of microbial species to co-exist in communities commonly referred to as consortia. These consortia function as integrated populations in which their common goals are sustenance, survival and dominance within their particular environment. Achievement of these goals involves cooperation between the species that can extend towards a synergy in which the total product exceeds the sum of the contributions of each of the component species. This synergy extends towards improved structural supports for the community through the synthesis of ferric oxides and hydroxides (such as goethites) and carbonates (such as hematites, siderites and dolomites). Vehicles for consortial colonization are also commonly generated by consortia in the form of particulate matter (colloidal or porous) that is released from the consortium in a form that enters the surrounding environment and increases the ability of the consortia to colonize the surrounding environment. Known examples of this are biocolloids that commonly form a part of the total suspended solids in waters, and red dust that is commonly produced by iron-rich bioconcretions.

The present invention is particularly concerned with the production of a therapeutic agent from a variety of consortial growths found in bioconcretions known as “rusticles”. Rusticles are a form of iron-rich bioconcretions that were originally discovered to be consuming the deep sea wreckage of the RMS Titanic. The rusticles were so-named based on their resemblance to rusty icicles dripping downward from the ship's wreckage. During a 1996 expedition, two hull fragments of the Titanic with such growths were brought up from the ship's debris field. Detailed study by the present inventors revealed the presence in a typical rusticle of a self-sustaining complex consortial structure having various means for retaining and transporting water, including iron-rich plate-like shells, bacterial threads and fibrillar bundles, all within a very spongy pumice-like pulp material.

As illustrated in the cross-sectional diagrams of FIGS. 1A and 1B, a rusticle is a complex assemblage of structure, displaying many characteristics normally associated with tissue differentiation in higher organisms. Each rusticle displays a series of microbial consortia dominated by bacteria and fungi which are, in essence, independent free-living cells grouped into differentiable structures. Included among these structures are internal water channels, water reservoirs, hardened iron-rich plates, surface ducts which pass through the external hardened plates and connect the rusticle interior to the outside environment, porous sponge- or pumice-like layers, bundled fibrillar clumps and elegant threadlike “girders” which appear to be strung through the structures and channels and apparently provide a measure of mechanical stability to the entire rusticle.

Since their initial recovery in 1996, rusticles have been artificially grown by the present inventors in a laboratory at the University of Regina on mild steel ingots, where they have formed complex attachment sites, by injecting a modified Winogradski's culture medium.

Detailed study by the present inventors has revealed the presence of over 20 different species of bacteria, at least two fungal species, and also members of the Archaea, cloistered in consortia strewn throughout the rusticle matrix. Although bacteria-like in appearance, Archaea is recognized as a distinct class of life all its own. In the rusticles, the Archaea appear to have entered into a symbiotic linkage with other organisms.

Although the precise nature of the consortia found in rusticles is difficult to precisely define, Dr. Roy Cullimore, one of the co-inventors herein, has endeavored to do so based upon a Microbial Consortial Identification Code (MCIC) comprised of a two-dimensional atlas containing many of the major bacterial genera. Each genus is located within the atlas in a manner that reflects the relationship of that genus to the neighboring genera. The MCIC is disclosed in Cullimore, D. Roy, “Practical Atlas for Bacterial Identification”, Lewis Publishers/CRC Press, Boca Raton, Fla. (2000).

To identify the various forms of consortia within the rusticles, a grid was superimposed on the atlas to enable identification of specific genera and provide the ability to identify a consortial growth by the shading or cross-hatching of boxes within the grid. In a section of this document entitled “Microbial Consortial Identification Code”, the principles of the MCIC and its application to the various forms of consortial growth found in rusticles are discussed along with illustrations of such consortia.

### SUMMARY OF THE INVENTION

The present invention relates to the formation, testing and use of microbial consortia and, more particularly, to the production of a mass culture of a consortia for the purpose of generating a desired product, to methods for the optimization, refinement and extraction of the product, and to methods of use of the product as an anti-microbial, anti-cancer, or other therapeutic agent.

Accordingly, the present invention relates to properties and products of consortia of microorganisms, e.g., communities formed of a plurality of strains of microorganisms, in which the collective of these strains generates products and engages in activities that do not occur without the presence of the plurality of strains and their associated activities. In accordance with the present invention, collections of multiple strains of microorganisms found in rusticles, referred to herein as “consorms” or “consortia”, are artificially cultured to generate agents of value that are extracted. In the various examples disclosed herein, the agents of value include anti-microbial agents, antibiotics, anti-cancer agents, and microbial stimulants.

The present invention is based upon the general characteristic of consortia and, in particular, the unique characteristic of those consortia found in rusticles, by which they dominate their local environment and prevent competition through the synergistic synthesis of antimicrobial agents that fall into the class of antibiotics or anti-cancer agents, and by which the consortia found in rusticles maintain dominance by selected groups of cells associated with the integrated compatible species that may also lead to the generation of non-integrative cellular intrusions. As demonstrated herein, the specific form of consortia found in rusticles have the ability to retard and eliminate cellular intrusions which would otherwise cause cancerous-like growths and degrade the sustainability of the consortium.

In particular, the present invention relates to various aspects of the production and use of specific microbial consortia, including the production of a mass culture of a specific consortia in order to generate a desired product, optimization of the defined product, product refinement and extraction, and use of the desired product.

The desired product could be a part, or a complete consortial structure. Alternately, and depending upon its efficacy, the desired product could be refined from the consortial structure or soluble/particulate/colloidal products released by a consortium during stages of growth and/or processing. This may include viable or inanimate cells from some or all of the species inherently a part of a consortium.

Preferably, electrical charge is used to focus biological activities and/or entrap the products of that activity within semi-saturated or saturated porous media, within water or colloidal structures attached to or suspended in that liquid medium, and to entrap product in downstream refined or raw effluent. Further, one of the co-inventor's patented Biological Activity Reaction Test (“BART”) systems, described in greater detail below, is used to identify the products of interest (e.g., antibiotics), optimize the production process, define the methodology for refinement and conduct the necessary quality control programs inherent in a well managed manufacturing and pharmaceutical plant.

### BRIEF DESCRIPTION OF THE DRAWINGS

FIG. 1A is a vertical cross-sectional view of a hanging rusticle showing a central water channel with saclike extensions into the porous cortex thereof;

FIG. 1B is a cross-sectional view of the structures found associated with the central water channel of the rusticle shown in FIG. 1A;

FIG. 2 is a perspective view of an apparatus used for growing a mass culture of a consortia;

FIGS. 3A and 3B are diametrical cross-sectional views of a culture chamber having a test device therein; and

FIGS. 4A-4H are illustrations of an atlas and grid used to identify the nature of various microbial consortia.

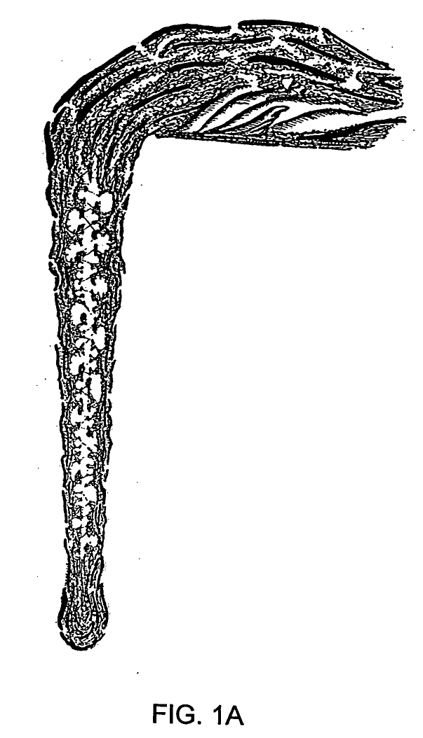

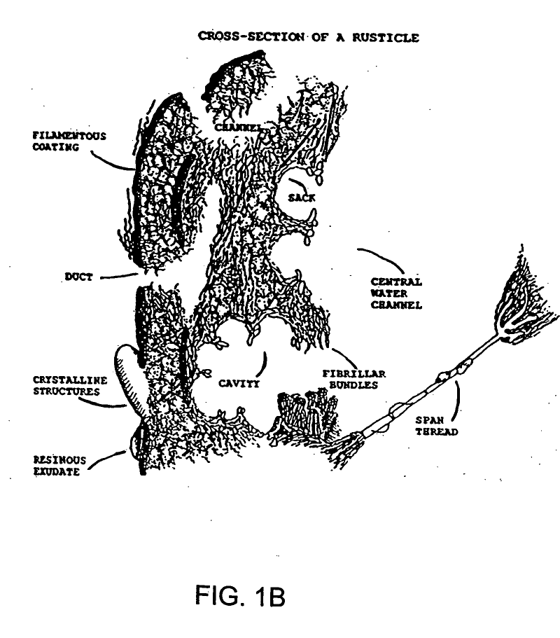

### DETAILED DESCRIPTION OF THE PREFERRED EMBODIMENTS

The present invention broadly relates to the general ability of microbial consortia to collectively produce anti-microbial compounds and other compounds of therapeutic or commercial value. The following description of a process for the growth of a microbial consortium and the refinement of an anti-microbial or anti-cancerous compound from the growth and/or activity of the microbial consortium is based on the activities of microbial consortia particular to rusticles. However, as will be appreciated by those skilled in the art, the invention is not limited solely to rusticles, but is applicable to any form of microbial consortia capable of use as or of producing products of value using the principles of the present invention.

The following detailed description is provided in four separate sections, including: (1) Production Methodologies—which discusses various methods for the commercial production of a selected microbial consortium; (2) Processes for the Bio-Magnification of the Significant Compounds—which briefly discusses processes for the bio-magnification of significant compounds generated through the activities of the selected microbial consortium; (3) Mechanisms for the Extraction, and Purification—which briefly discusses mechanisms for the extraction and purification of antimicrobial and/or anti-cancerous compounds; and (4) Microbial Consortial Identification Code—which discusses the inventors' method of classifying the nature of various consortia found in rusticles.

1. Production Methodologies

Microbial consortia generally thrive in specific habitats. In the present description, emphasis will be placed upon the culture of rusticles in an aqueous solution that simulates the constituency of sea water. As will be recognized by those of ordinary skill in the art, this does not exclude the potential to adapt the principles disclosed herein to the production of microbial consortia in various other habitats such as in porous media, at various interfaces, and within or upon giving systems.

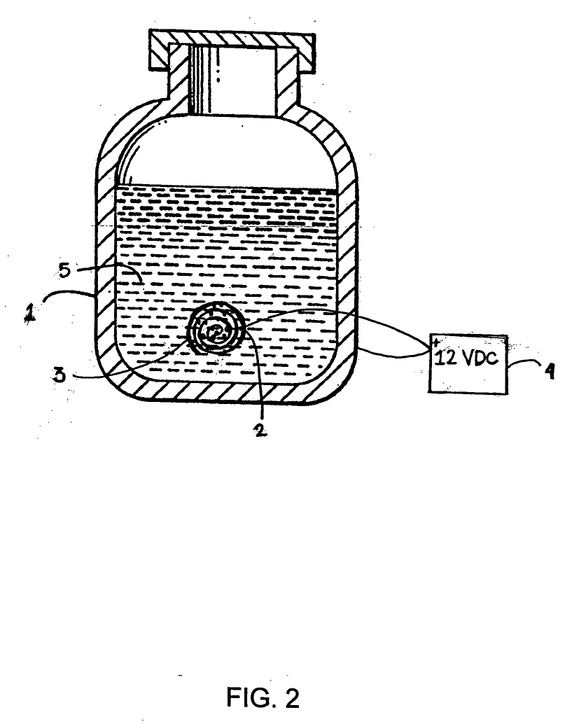

Referring now to FIG. 2, in accordance with the presently preferred embodiment, the rusticles are grown in a tank 1 from “seeds” 2 comprised of small samples of rusticles such as those originally collected from the debris field of the Titanic. The seeds are placed on a growth substrate 3 comprised of surfaces of anodically charged metal or plastic materials designed to allow the attachment, growth, and production of the desired anti-microbial material.

In the presently described embodiment, the rusticles were grown on a growth substrate 3 formed of a 10 mm mild steel plate. Prior to use, the steel plate was embrittled by a 10% hammering of its surface at about 3000 p.s.i. for approximately 60 minutes. This treatment provided an embrittled surface into which the rusticles were able to penetrate and form initial bio-concretious structures. Of course, the growth substrate and treatment methods are widely variable. Those given herein are to be taken as being merely illustrative. As will be appreciated by those of ordinary skill in the art, the embrittling conditions described above, as well as the use of a mild steel growth substrate, are merely examples. Many different types of pre-treatment methods, or no pre-treatment method, may be employed and the growth substrate may be formed of any material on which bacteria are likely to grow.

In the presently described embodiment, the mild steel plate employed as a growth substrate had a chemical composition including a minimum of 0.8% phosphorus, 1.2% sulfur, and 1.4% manganese. The steel plate was rolled in such a way as to ensure that lateral pearlite deposits were formed within the steel to cause a flaking form of corrosion. An anodic charge was applied across the steel plate using a 12 volt DC power supply 4 with an inert cathode. However, any electrical charge that is deemed appropriate can be used. Alternatively, if a growth substrate formed of unlike mating metals such as Fe and Cu is employed, no electrical charge is necessary to stimulate rusticle growth.

The growth of the rusticles on the charged surfaces occurred around the sacrificial anode and formed structures which cling to the steel plate and also formed gas-rich deposits on the floor of the tank 1 in which the growth occurred.

The electrical current may be applied appropriately based on the need dictated by the process. By way of example, charge could be administered for five percent of the time in a pulse like manner with, for example, five minutes on and 95 minutes off.

The apparatus for culturing the rusticles is submerged in water 5 containing a minimum of five ppm of oxygen, a nutrient substrate containing ferric ammonium citrate at 1 percent and inorganic nutrients and levels typically used for the culture of microorganisms. The pH was allowed to fluctuate within the range of 7.4 and 8.4 and the salt concentration was maintained in the range of 0.3 to 0.8 percent using a typical sea water mineral composition. Phosphorus was controlled to be within the range of 0.05 and 0.15 ppm. The temperature of the culture was maintained in the ambient room temperature range but may be adjusted appropriately for optimization of the process.

Using the above-described conditions, significant growth of rusticle-like structures was seen within seven days. Within that time, the rusticles began to release a red dust at a rate between 0.015 and 0.02% of the total body of mass of the rusticles per day. Various anti-microbial activities of interest are focused and concentrated within this red dust. Since the dust has a density between 1.4 and 1.8, it is easily separable by passive sedimentation or active centrifugation.

However, as will be readily appreciated by those of ordinary skill in the art, the presence of beneficial properties in the red dust certainly does not preclude the likelihood of additional compounds of value generated and released into colloidal structures in the aqueous medium or dissolved into the medium itself.

By experimentation, the present inventors have discovered that the growth rate and cycle of the rusticles is dependent upon the supply rate of nutrients, electrical power, and the presence of a sacrificial anodic metal such as mild steel (or unlike metals which simulate the application of a voltage in the aqueous solution). Therefore, the present invention is not limited to the details of the growth method described above, but the potential to culture in mass amounts microbial consortia in any appropriate media and form, wherein the activity of the consortium generates a desired product that has any form of anti-microbial, anti-cancer, or any type of bio-inhibitor of potential value to humankind.

As pointed out above, while the examples used herein to illustrate the present invention relate to the form of iron-rich bioconcretions commonly referred to as rusticles, rusticles provide only one example of a consortial form of microbial growth. The present invention is intended to encompass various different forms of consortial growth, including the various forms of slimes, natural crystal and noble metal formations, tubercles, nodules, encrustations, reduced forms of hydrocarbons (such as anthracenes, paraffins, oils and coals), foams and suspended colloidal structures. All of the foregoing types of structures, as well as any others that are not identified herein, include growth and/or development attributable to the activities of microbial consortia. Moreover, as will be readily appreciated by those of ordinary skill in the art, in all of the foregoing cases, the application of electromagnetic force to create charged locations could lead to managed focusing of the microbial consortial activities. These consortia would normally consist of a mixture of species commonly including various fungi, iron related bacteria, sulfate reducing bacteria, slime forms, denitrifiers, heterotrophic bacteria, nitrifiers, fermenters and methane generators. The exact nature of the consortium would be created by the nature of the electrical environment achieved, the nutrients applied and the physical conditions applied. The desired product may be in the form of the harvested consortial mass, products recovered and/or released from the mass, or accumulated products of the activity. The product of value would be a construct from the consortial activity that would have the desired impact. This may take the form of an animate or inanimate selective agent which has been generated by the consortial activity and has a suitably manageable negative (e.g., antibiotic, anticarcinogenic) or positive (e.g., hormonal, nutritional) effect.

2. Processes for the Bio-Magnification of the Significant Compounds

As will be appreciated by those familiar with the art, there are a number of known techniques for the recovery of anti-microbial compounds. The techniques described herein involve features that are uniquely applicable to microbial consortia. These techniques include the use of electrical potential anodic biological biofocusing, the stimulation of the microbial consortium to synergistically generate optimal amounts of the selected compound of interest within the form and conditions of the environmental mass culture device, and the secondary but significant features that may be directly associated with the form and/or function of the consortial biomass.

The electrical potential anodic biofocusing technique (EPABF) is a phenomenon in which the various animate and inanimate products of growth can be relocated by creating a selective electromagnetically induced charge gradient across suitable materials such as embrittled steel, graphite carbon, clays, and other porous or solid materials capable of carrying, and preferably holding, electrical charge.

In a preferred embodiment of the present invention, the application of EPABF is schematically illustrated in FIG. 2 by the power source 4 having an anode connected to the tank 1. EPABF is applied downstream of the culturing device to the surfaces of the tank 1 to create the primary mechanism for the product recovery, whether in the form of completely dissolved molecular material, materials that are trapped within a bio colloidal matrix, or as porous or solid suspended particulate materials. In the latter case, it is probable that the material would be functionally biological and behave as primarily negatively charged entities. In these cases, an EPABF gradient would focus the attachment of the particulate materials to anodic interfaces.

As will be appreciated by those of ordinary skill in the art, the ability to manipulate an EPABF gradient to cause a manipulated and predictable relocation of the targeted organisms or materials of interest has far-reaching implications beyond the simple concentration of the product. For example, the ability to electrically induce the movement of pathogens, nuisance bacteria, or non-conforming tissue cells (e.g., cancerous tissues) has widespread implications and applications.

Another method for optimizing the production of a desired agent is by controlling the environmental circumstances which are natural prerequisites to the successful generation of a consortia growth. The culture of a rusticle consortium involves careful control of various factors including the surface form and composition, the electrical charges in the environment, the form of the feed stock (e.g., bio-colloidal, dissolved, solid particulate, embrittled porous, non-porous solid, the hydraulic flow patterns, temperatures, pH, redox potential, salt concentrations and the surface area volume ratios. In order to optimize the product mass, it is essential to predetermine the conditions necessary to maximize the functioning of the consortium to produce the product. This is a matter of routine experimentation. Notwithstanding the need to optimize, it should also be recognized that consortial systems operate in harmonic patterns which may cause the culture system to be most useful when generated in batches (using a fermentor) with the sequenced production of product. The design and form of the device would reflect the form of the consortial structure being nurtured in a manner to optimize the production of the selected consortially generated product of choice.

3. Mechanisms for the Extraction and Purification

At this time, there is evidence of the anti-microbial activity of the rusticle consortia against numerous bacteria, including pathogenic bacterial species, Pseudomonas aerugirosa. The nature of the inhibitory effect appears to be linked to either (or both) the generation of an anti-microbial compound synergistically synthesized by the consortium as a whole, or the very high sorption characteristics of the rusticle when it is released in the form of a red dust. Therefore, the present invention is directed to the collective generation by a group of bacterial species of a singe anti-microbial compound to achieve dominance within the environmental niche being colonized. The present invention is also directed to the ability of the consortium operating in a porous very sorptive matrix to out-compete competitors including pathogenic bacteria.

There are two mechanisms that appear to be primary driving forces in the suppression of pathogenic bacteria by the consortial growths and/or that product. These mechanisms are (1) members of, or the consortium as a whole, being able to out-compete the pathogenic bacteria at the infection sites and, in doing so, reducing the symptoms of that infection to sub-clinical levels; and (2) the ability of the consortium to provide such extreme, and attractive, sorptive sites to the pathogenic bacterium that these cells are sorbed into the consortial matrix and neutralized.

The determination of the presence of antimicrobial activities was achieved by use of the previously patented BART bio detectors utilizing computer-assisted monitoring hardware and software. The patented BART biodetection system generates a complex of nutrient and redox gradient within which many more bacterial species and consortia are able to become active and be detachable. This methodology is more sensitive (because of the variety of environmental niches presented) and capable of detecting a broader spectrum of bacterial activities than can be achieved by the conventional agar plate and broth culture techniques. As a result of this improvement, the opportunity exists to detect anti-microbial agents more effectively, more economically, and over a shorter period of time.

More specifically, Dr. Roy Cullimore, a co-inventor herein, is also a named inventor in U.S. Pat. Nos. 4,906,566; 5,187,072; 5,531,895 and 5,589,353, each of which is incorporated herein by reference in its entirety. These patents disclose methods and apparatus for the production, growth and determination of fermentive analytic cultured activities in a given system, referred to herein as the BART system.

For instance, U.S. Pat. No. 5,187,072 discloses a test system for testing for the presence of selected microorganisms. A sample is mixed with a culture medium for promoting growth of the selected microorganisms. A test device comprised of a semi-permeable material is inverted in the culture medium and the test device is held in a vertical position. The microorganisms create a biofilm within the test device, and the test device subsequently retains gas generated by the microorganisms. The gas lowers the density of the test device, causing the test device to float. The floating is taken as an indication of the presence of the selected microorganisms. By measuring the time required for the test device to float, the size of the population of the selected microorganisms can be determined.

In the patented BART system, biofilm formation reduces the permeability of the test device to chemical and biological entities so that the test device becomes a repository for gases generated by the incumbent biochemical activities. As a result, the test device, originally slightly more dense than the culture liquid, now has a reducing density as the gases form within the device. At some point in the diminution of density, the test device will begin to float and will move to a vertical position. With further gas generation, the test device will truly float, and will elevate to the surface of the liquid culture medium. The repositioning of the test device may be recorded visually after a standard period of time for a presence or absence determination. Alternatively, the precise time of the repositioning event may be recorded by such means as visual-image analysis or shifts in the conformity of a physical pathway such as a light beam. In the latter events, the delay time may be projected and the possible population of targeted organisms calculated statistically. The result is that fermentative gas producing biological events can be readily monitored in a given culture liquid and qualitative or quantitative interpretations appropriately made.

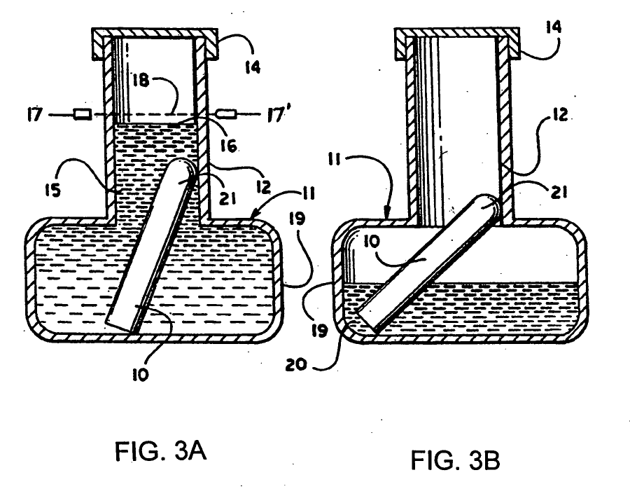

A preferred embodiment of the BART system is illustrated in FIGS. 3A and 3B of the drawings. One mechanism for quantifying the population of the target group of bacteria is to time the delay period to a specific physical relocation event of the device. One such example is to measure the time of delay before the device interrupts a light beam. As shown in FIG. 3A, the light beam 18 is propagated by a light source 17 on one side of a test chamber 11 comprised of upper and lower cylinders 12, 19, and received by a light detecting means 17′ on the opposite side of the test chamber 11. The test chamber 11 is structured in such a way that the test device 10 cannot slide down to lie laterally on the floor of the chamber. The test device 10 must remain sufficiently vertical to allow the entrapment of any gas generated within the test device 10.

As illustrated in FIG. 3B, prior to the charging of the chamber 11 with the liquid sample, the vessel will contain a concentrate 20 of a selective culture medium which will, upon dilution with the liquid sample, cause standard cultural conditions to be created in order to support the activity of the targeted organisms. At the same time, the test device 10 will not be totally immersed in the concentrated culture medium 20 but will extend into the atmosphere above the liquid. Some of this atmosphere may therefore become entrapped within the device and have to be expelled prior to the initiation of a test procedure.

The test device 10 consists of parallel walls and a hemispherical dome 21 at one end. Both the walls and the dome 21 are constructed of semi-permeable material to form a porous medium through which microorganisms can pass, and to which sessile organisms attach and form biofilms. The significant surface area presented by the device 10 will allow a rapid colonization of the porous medium with occlusion occurring. The construction of the structural walls of the device therefore will allow initial passage of organisms through the walls of the device and into the interior of the device 10 which acts as a reaction chamber. Once occlusion has occurred, gases generated will be retained within the device 10.

When the device has now become coated with an expanding biofilm as cultural activities develop, the device 10 occupies a vertical position with the domed end 21 uppermost, facing the air-liquid interface 16. As the cultured activities develop within the test device 10, any gas generated may rise to become entrapped under the dome 21. The initial effect will be to cause a loss in density so that the device will assume a vertical position.

### EXAMPLES

The following examples are based on various forms of consortial activity. The first example uses the rusticle as the prime consortium in which there is a consistent production of rusticle red dust (RRD). As noted above, the RRD can be produced at passive rates equivalent to 0.015 to 0.02% of the weight of the biomass per day and commonly has a density of 1.2 to 1.4 with a mean diameter of 40 microns. When anodically stimulated, the rates of production increase minimally by one order of magnitude, which represents a considerable increase in production.

It has been confirmed by applicants that the RRD, like the rusticles, does cause sorption (using methylene blue as an example) and has a surface area commonly found to be approximately one third of that of granulated activated carbon. Such strong sorptive characteristics have been found by applicants to have impacts on the forms and rates of growth of the bacterial cultures in various ways. For example, RRD was observed to grow out and extend over bacterial colonies as a (1 to 3 mm) growth of slime which originated around the RRD particles placed on the agar surface. A typical example of this was the impact on a species of Micrococcus. Here, the RRD slime formed firstly around the outside of the bacterial colonies to form haloes 1-2 mm wide. Upon the total coverage of the colonies with the slime, the colonies disappeared and were presumably cannibalized by the slime generated by the RRD particles. There appeared to be a direct interaction between these slime growths and the infested colonies but at the same time, there were no obvious inhibitory zones around the slime growths suggesting that no diffusible antibacterial materials had been released. Slime extensions also grew around Pseudomonas aeruginosa with the extensive slime growths moving out across the agar for distances of up to 30 mm. It was therefore projected that the resident population of slime forming bacteria in the RRD had the capacity to compete with these pathogenic bacteria in an agar medium environment. The mechanisms of control (of potentially pathogenic microbial and other cellular forms of growths) would therefore be a combination of suppressive activities resulting from the generation of inhibitory materials, the out-competing of the infesting organisms and/or cells for vital nutrients, oxygen and protective niches, and also the aggressive sorptive function of the RRD that would limit the availability of dissolved and particulate supportive chemicals to the infesting units.

Using the patented BART system described above, applicants found that respiration function of Pseudomonas aeruginosa was reduced for at least 6 to 18 hour period in the presence of RRD. At the same time, the growth of the RRD generated six months and two years previously was stimulated. Staphylococcus epidermis was totally inhibited in the presence of the RRD while the controls (free of RRD) gave a normal rate of growth for those conditions. Salmonella typhi caused an interaction with the RRD and the bacteria causing a blackening of the basal nutrient pellet displaying abnormal cultural growth patterns. When Staphylococcus aureus was tested, the RRD totally inhibited the respiratory function. Streptococcus pyogenes, when exposed to the RRD, did not grow but the respiratory function did not appear to have been affected in the early stages of the exposure. The RRD also prevented both the growth and the respiratory function of Micrococcus Iuteus. In addition, the RRD resulted in a 20% delay time in Escherichia coli and a 60%; delay time in Staphylococcus epidermis.

From these studies, it was determined that the newly grown RRD (less than six months old) appeared to have the greatest impact on a wider range of the bacteria tested and could be related to a direct competition between the RRD strain and the pathogens, the losses of sorption sites available to the pathogens due to competition with the RRD, and the generation of anti microbial agents (e.g., antibiotics) by the consortia of microorganisms involved in the RRD. It was also observed that the bacteria within the RRD consortium could grow out of the RRD and directly compete with, and parasitize, the pathogenic bacteria being tested.

The bio-concretious structures employed for the study included not only the red dust (RRD) generated from the laboratory growth of rusticles, but also whole fragments of deep-ocean rusticles themselves as well as yellow colloidal materials generated during the growth of rusticles within the apparatus. Determination of the inhibitory nature of the product was determined specifically for a range of five American Type Culture Collection (ATCC) strains of bacteria. Evaluation of the inhibitory effects of the consorm-generated product was observed using the time lag to reductive conditions in a heterotrophic aerobic bacteria BART system. The time lag to a reductive state illustrated the level of microbial activity with shorter time lags meaning greater microbial activity. The results for the RRD were discussed above. The rusticle fragments had a complete inhibitory effect on Pseudomonas aeruginosa (ATCC #27853) and Staphylococcus epidermis (ATCC #12228), and a partially inhibitory effect on Escherichia coli (ATCC #25922), Serraita marcescens (ATCC #8100) and Proteus vulgaris (ATCC #13315). The colloid had an inhibitory effect on Staphylococcus epidermis.

The present invention therefore represents a singularly unique and valid concept which would allow the more effective screening, production and purification of products generated by consortial microbial activities. As will be appreciated by those of ordinary skill in the art, the present invention goes beyond the simple generation of a single target-specified compound (such as an antibiotic) to a new approach to the manner in which nuisance and pathogenic microbial events can be controlled through manipulated challenges by these microbial consortia. In essence, the present invention enables the development of far more effective control mechanisms utilizing the activities of microbial consortia rather than single-strain species of organism.

Notwithstanding the examples above given for the RRD, these claims also apply to the other aforementioned consortial structures. Some examples of these are presented below and relate to the formation of a consortially driven nucleation of suspended colloidal particles of sufficiently small size and dispersed nature to form a cloud held in that state at least partially by electrical charges and low densities. The nucleation is achieved by the charges in the polymeric matrices generated by, and formed around, the microbial cells and acts to accumulate water molecules into a droplet that may remain suspended within a mist or cloud. Where the consortium construction is dispersed in nucleated droplets, there is the potential for the droplets to contain such inhibitory materials as may be necessary to reduce the risks of an alien infestation of the droplets that could compromise the nature of the consortium. For example, where the nucleated droplets carried a consortium including a species capable in association with the other species of producing an antibiotic similar to erythromycin. When the droplets are inhaled, some of the droplets with the consortial flora would be inhaled. As will be appreciated by those of ordinary skill in the art, a normal soft gentle breathing would involve twelve breaths per minute each with a tidal volume of 500 ml. This would mean that the inhaler would take in six liters of air containing the consortial droplets per minute. Since the droplets would interact with the highly capillarized tissues in the lungs, the probability for direct interaction between the consortium and infective pathogenic agents and dysfunctional tissue growths is enhanced with an increased potential for competitive interaction and control of the infestation.

Accordingly, the present invention is based on the fact that most microbial activities take place within communities involving several species that are co-dependent and frequently function in a synergistic manner. These communities are therefore consortial and frequently include mutually synthesized mechanisms to protect the consortium from infestation by hostile species. The prime focus of the present invention is that these defense mechanisms, when understood and appreciated, can be utilized to form a valuable array of manageable chemicals and processes that could be employed to reduce and eliminate infestations, infection, bacterial and cancerous growths in humans and other significant species.

4. Microbial Consortial Identification Code

Traditionally, identification of microorganisms has been performed at the level of a single microbial strain where it is assumed all of the composite cells within the culture being identified have a common genetic code and common set of physiological and biochemical characteristics. While this level of precise analysis allows confident declaration of a single strain of microorganism, it does not address the interaction that such a strain may have with other strains of microorganisms within its environment. It may therefore be observed that identification of microorganisms has essentially been at the cellular level. As a result, the development of the science and art of microbiology has been focused at the single strain level of functionality. The following discussion addresses a community-based approach to the classification of microorganisms given the possibility that many of the activities are the result of consortial activities resulting from interaction between the various component strains.

Definitions for communities of microorganisms are generally considered to be through the generation of consortia. A consortium is defined as an association that may be of several companies or, in this case, several species. Association would involve the act of harmonizing activities of the members of the consortium for the common betterment of all of the members. The validity of microbial consortia as major contributors within most environments is now becoming recognized as significant in the understanding of soils, waters, foods, industry and the medical arts.

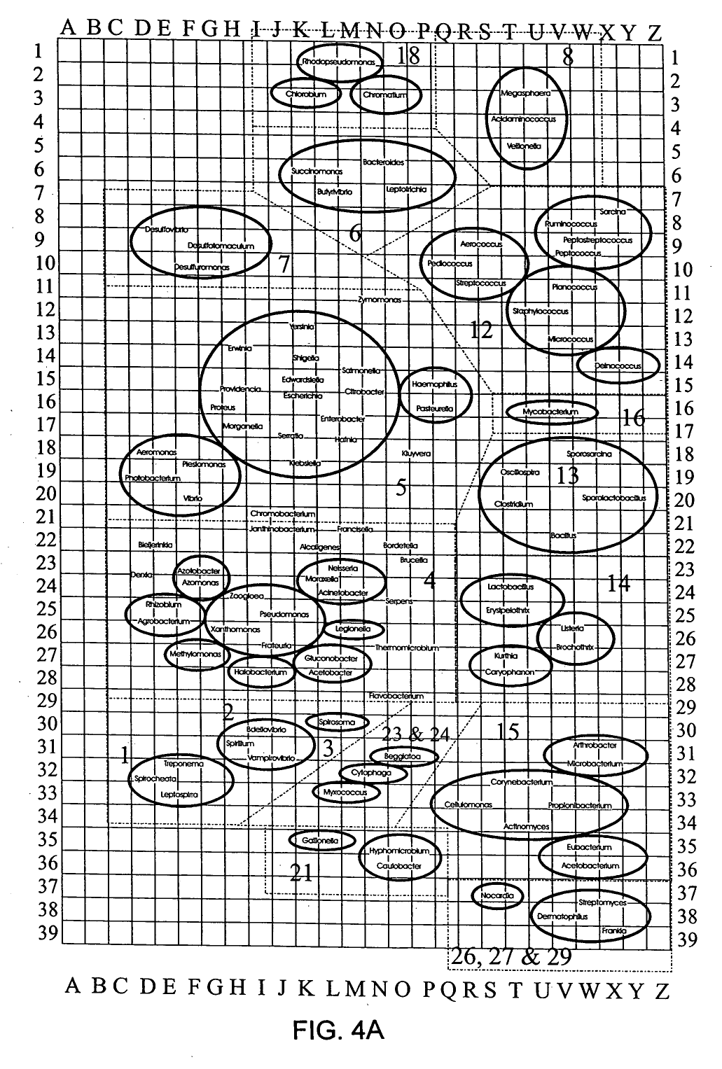

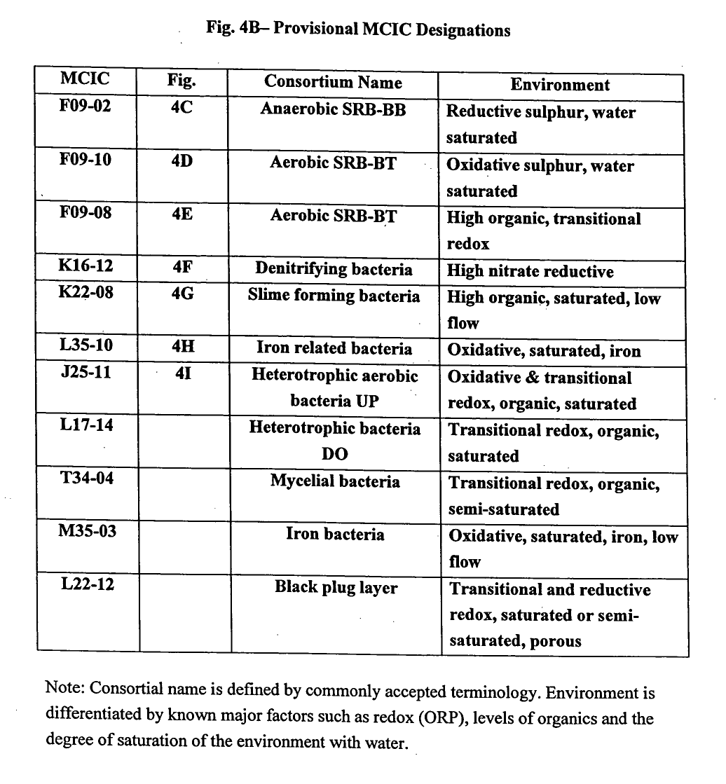

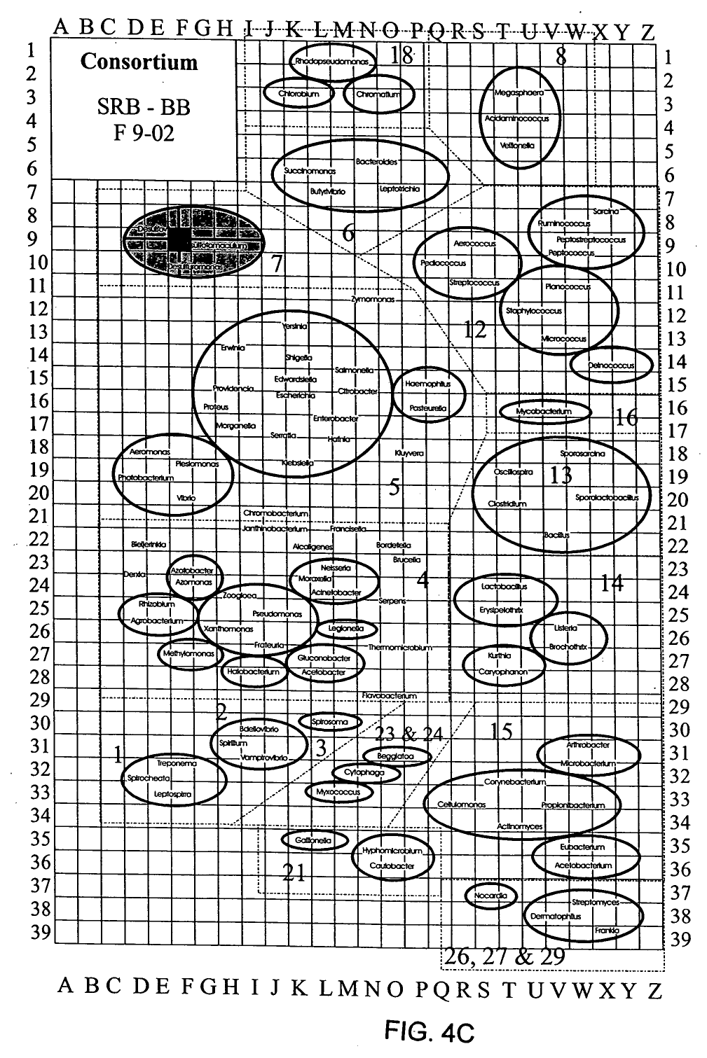

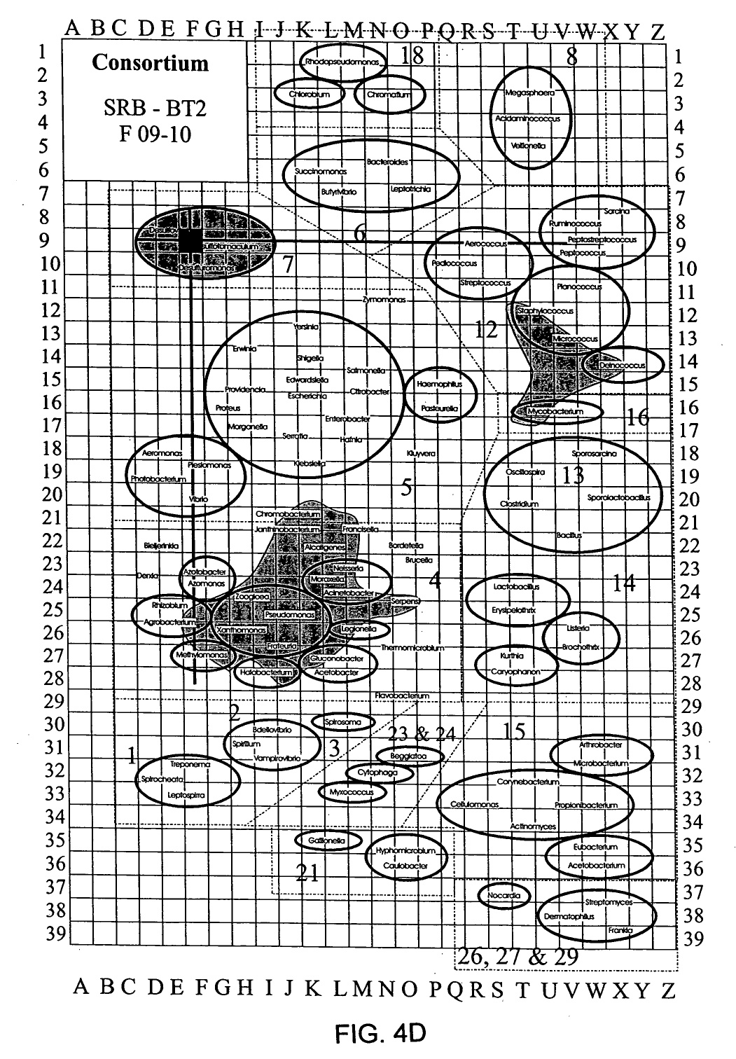

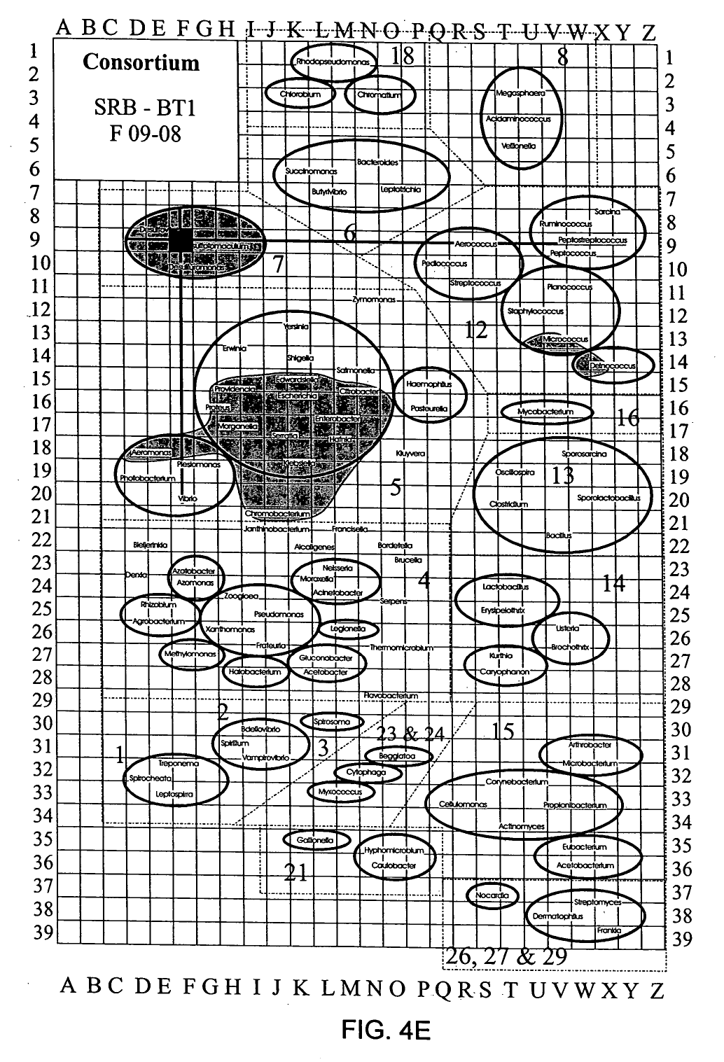

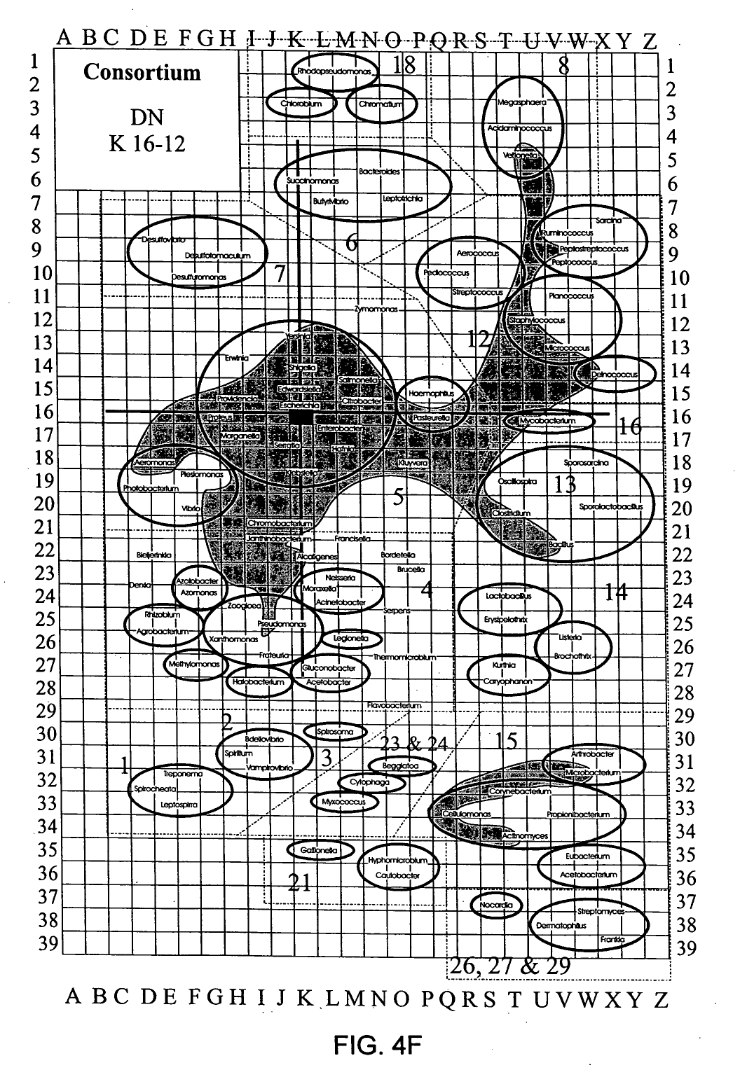

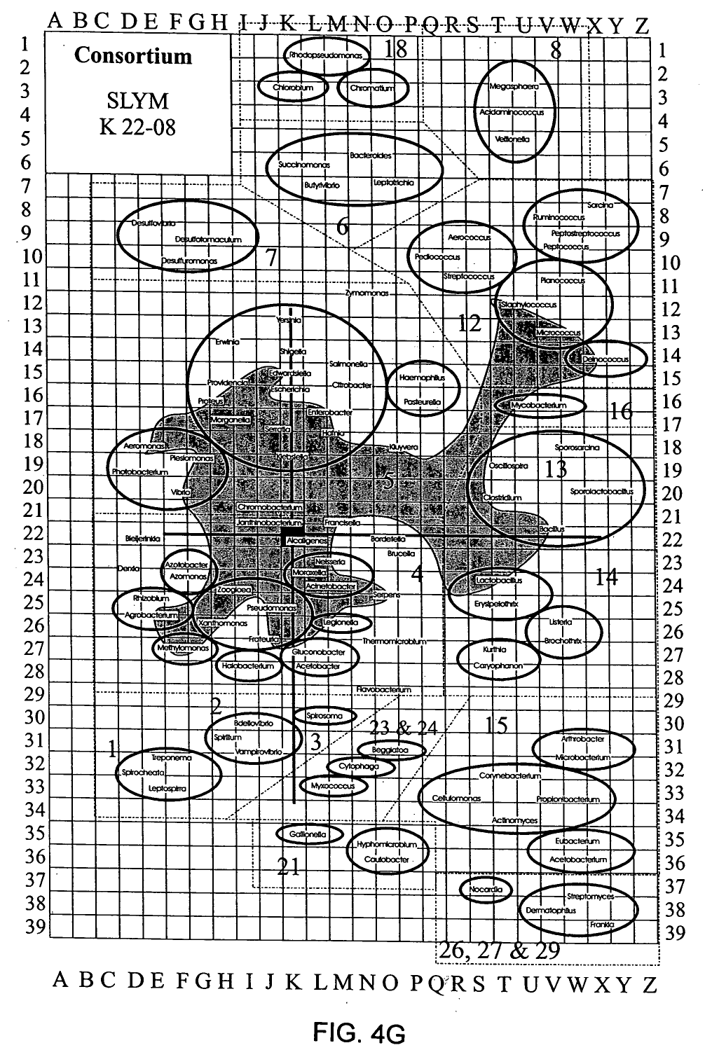

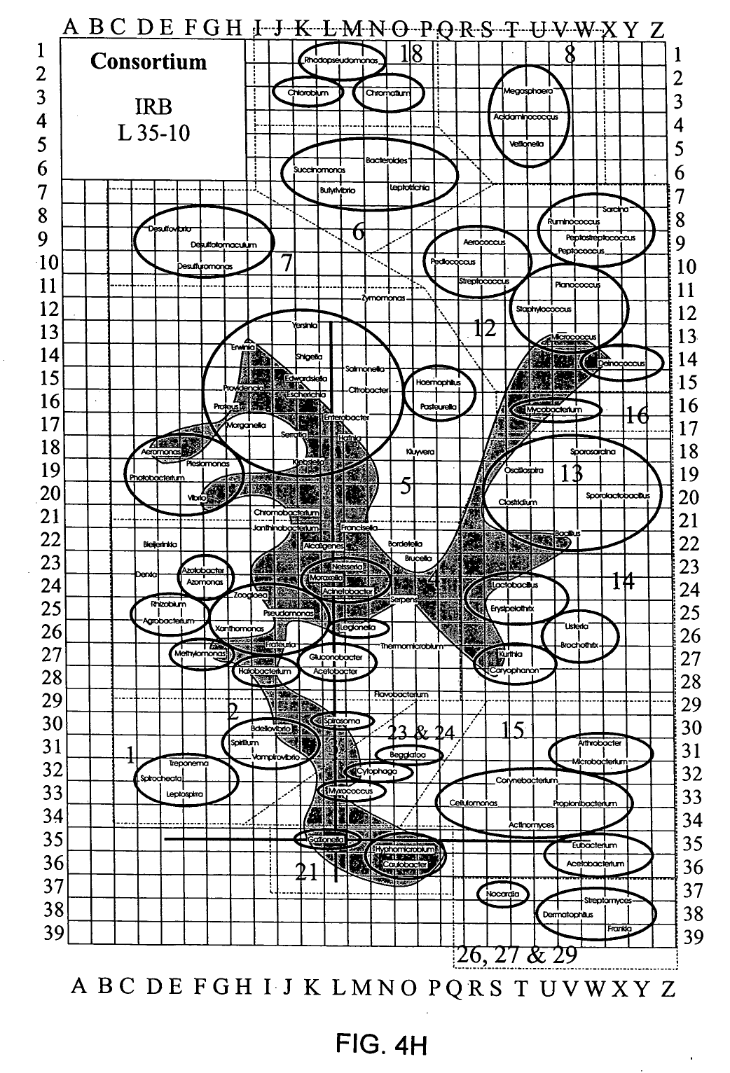

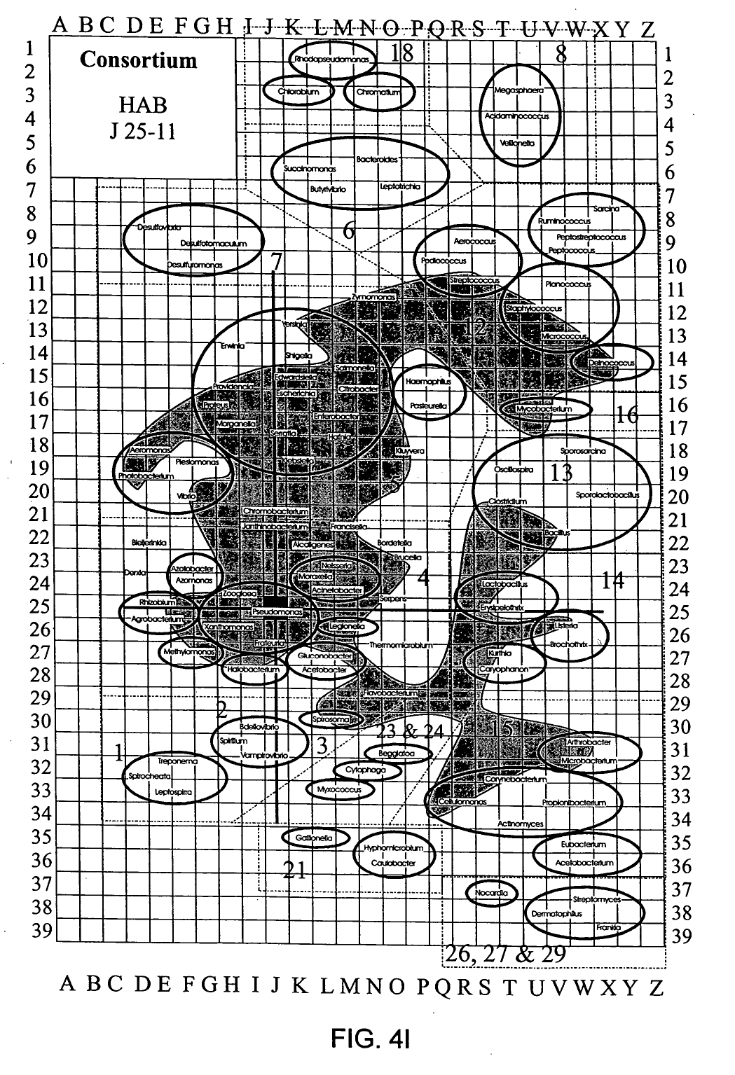

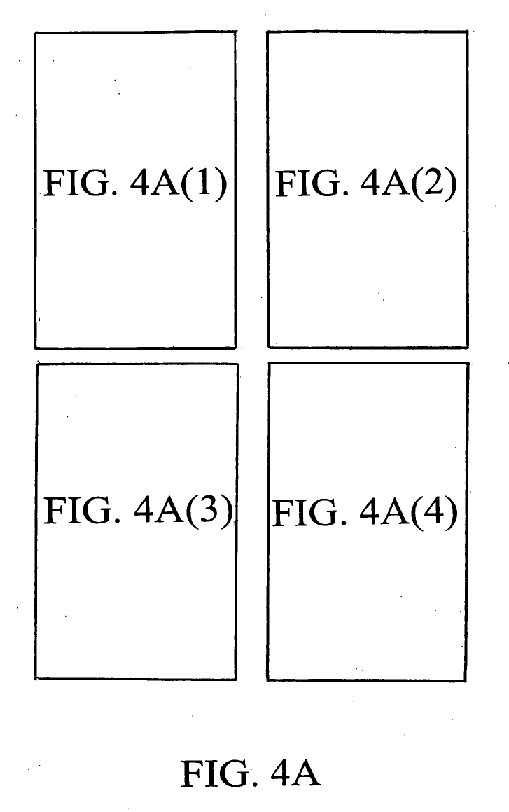

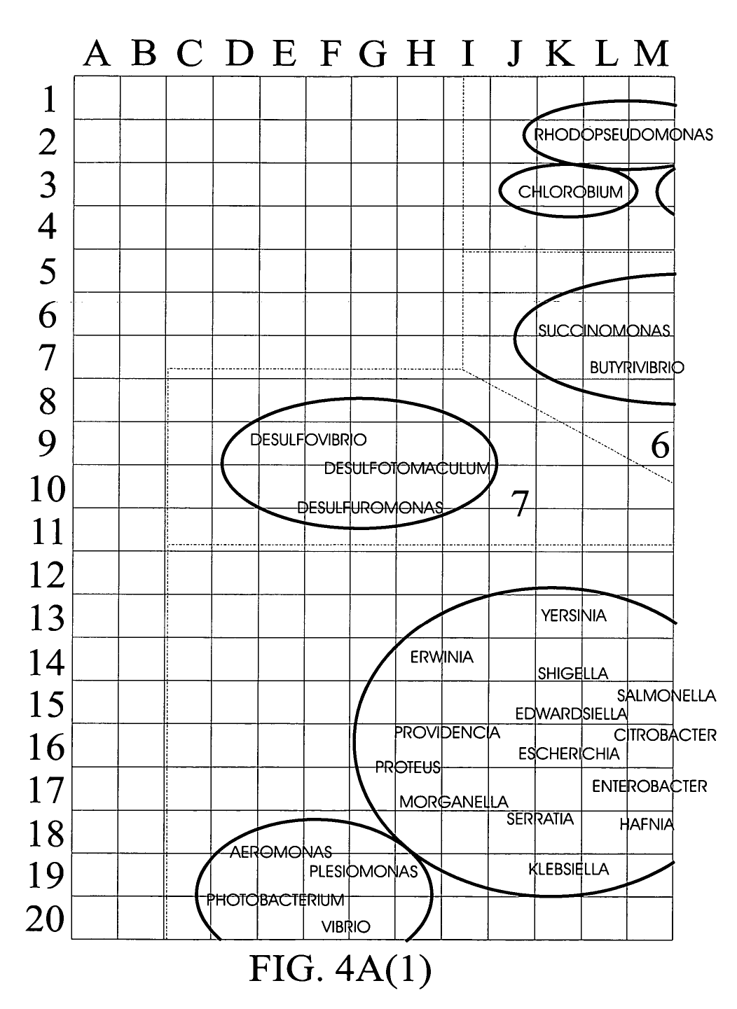

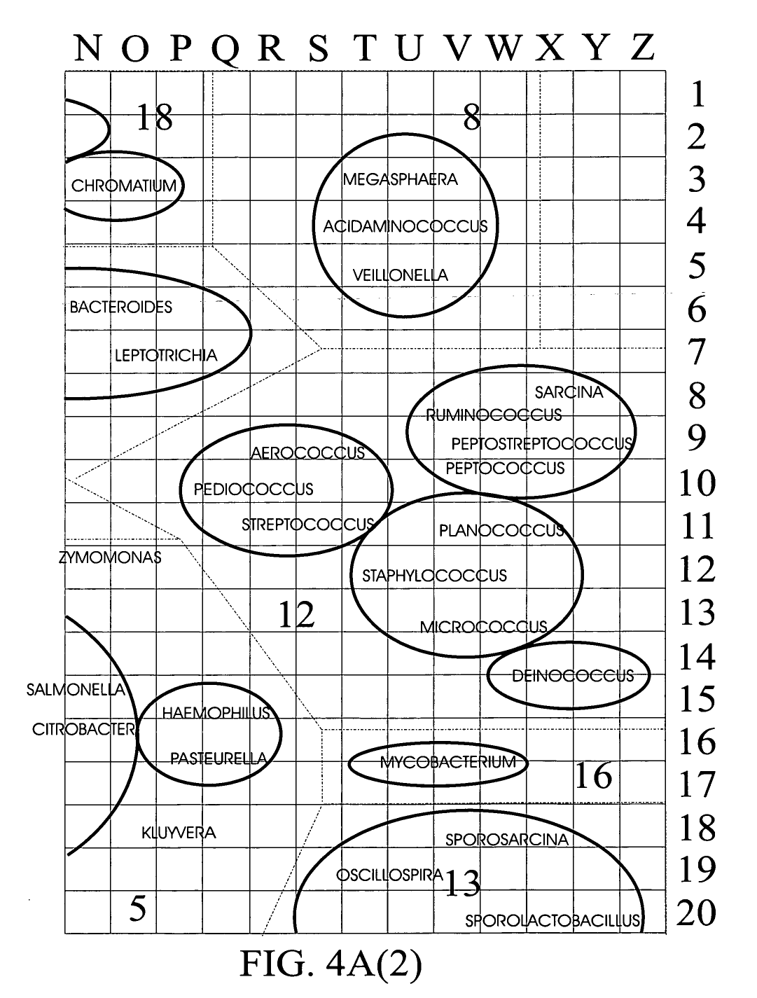

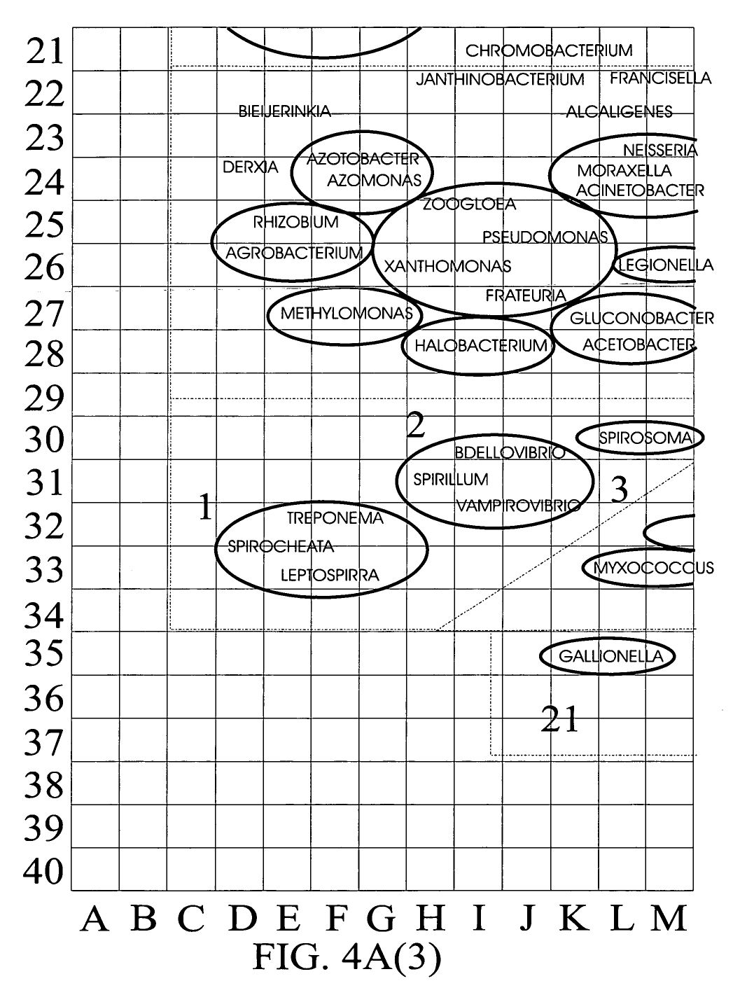

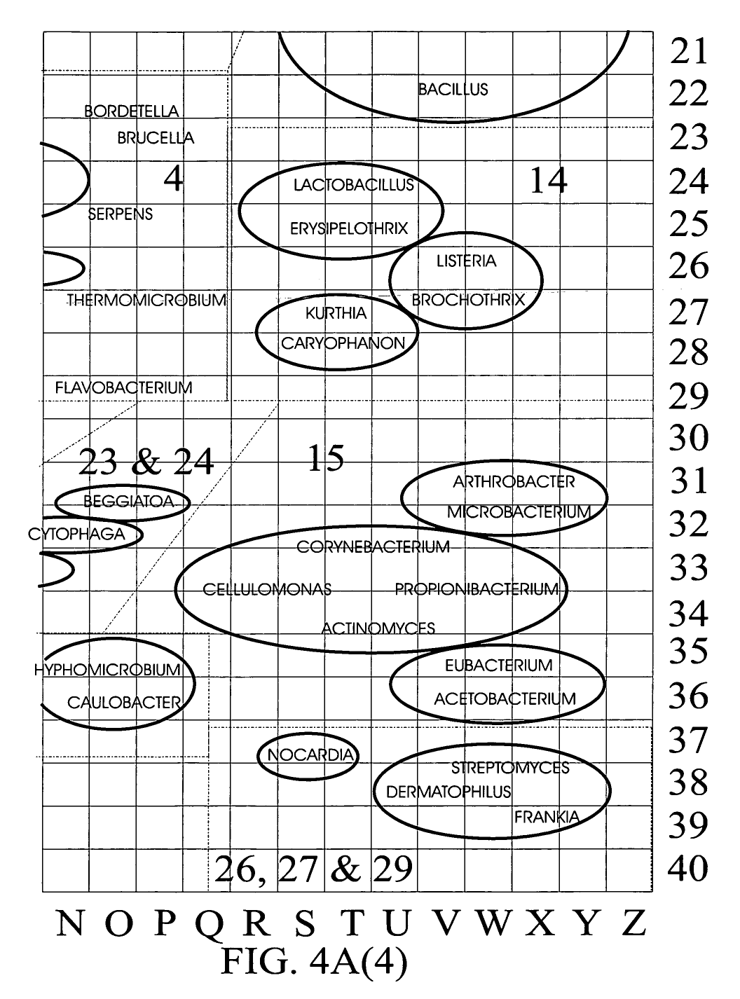

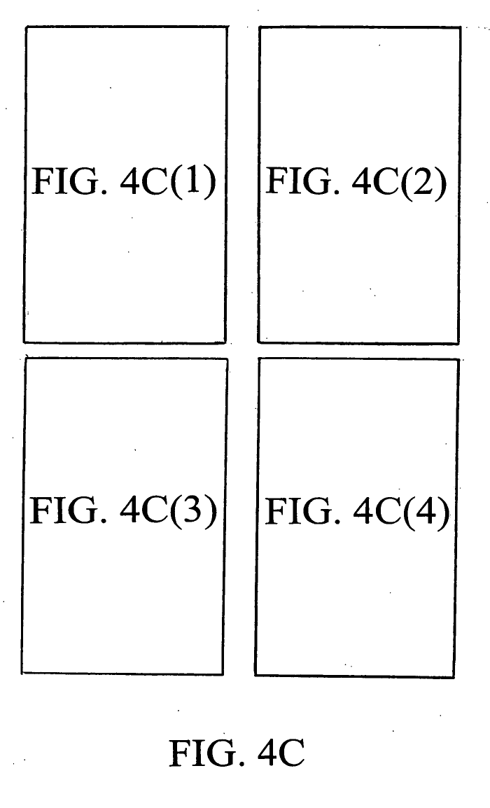

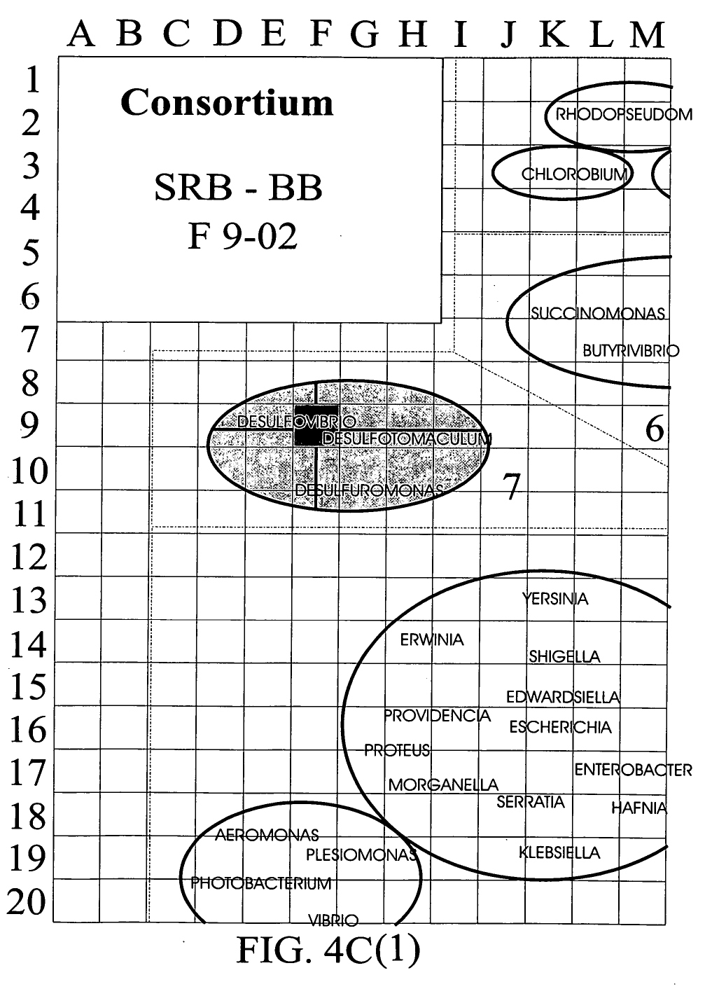

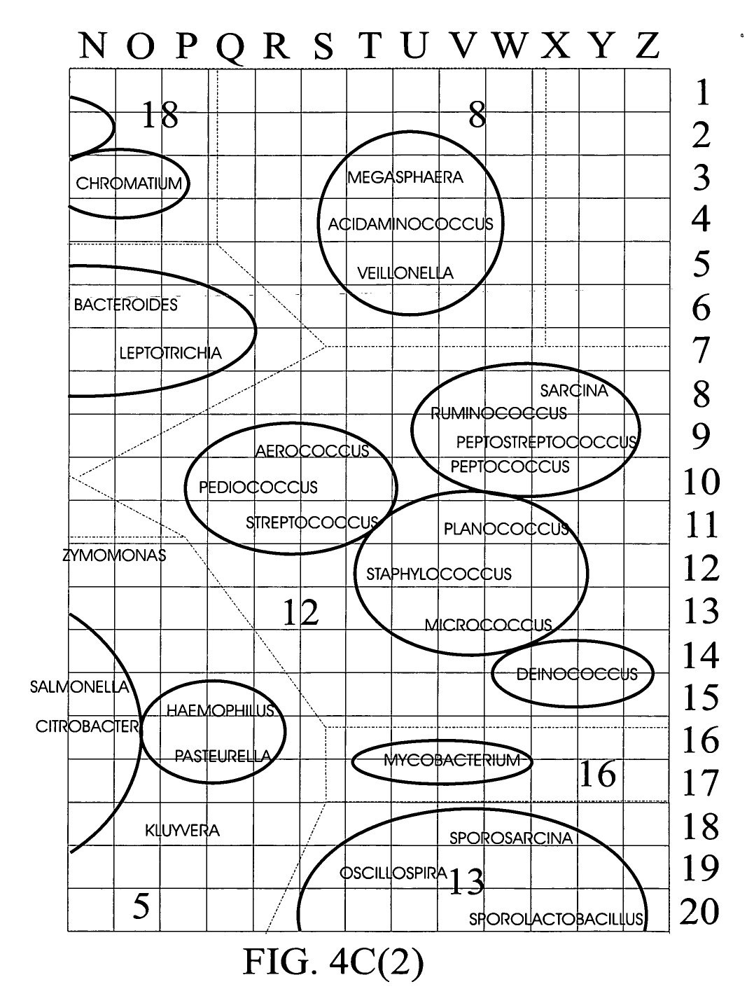

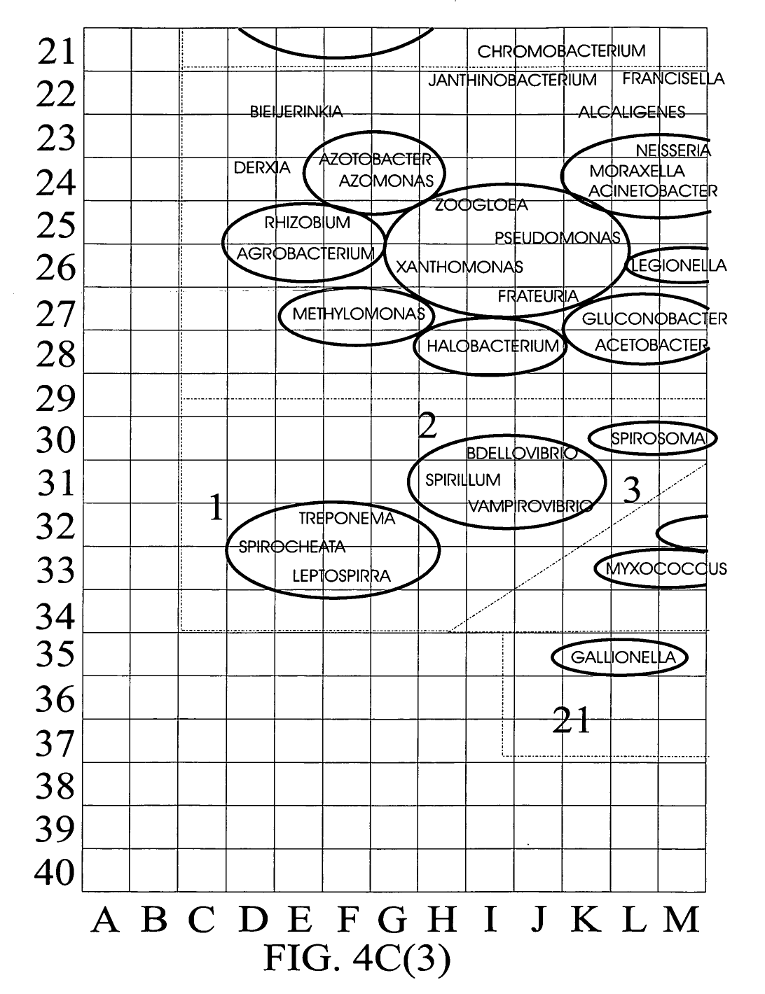

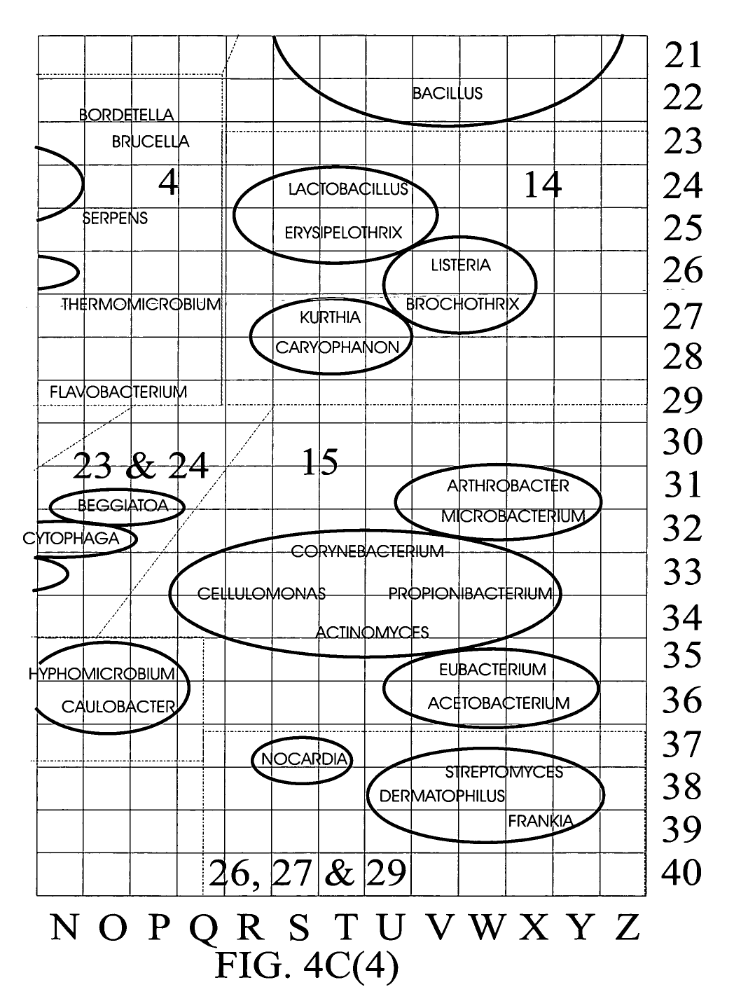

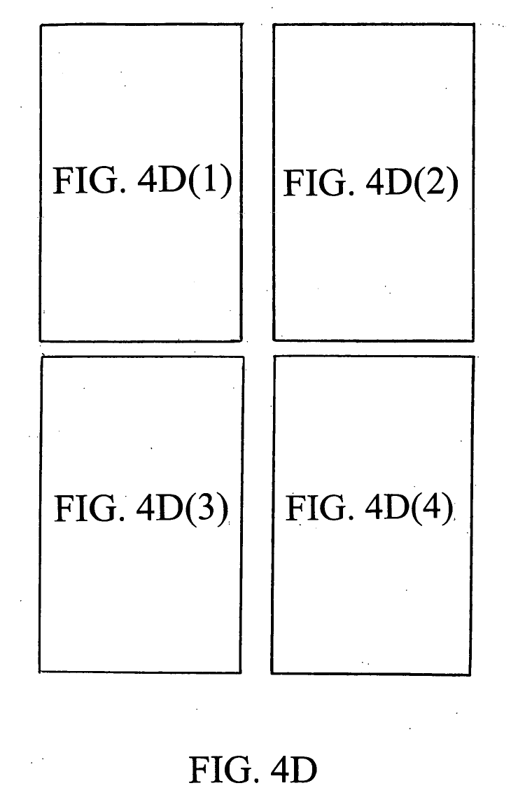

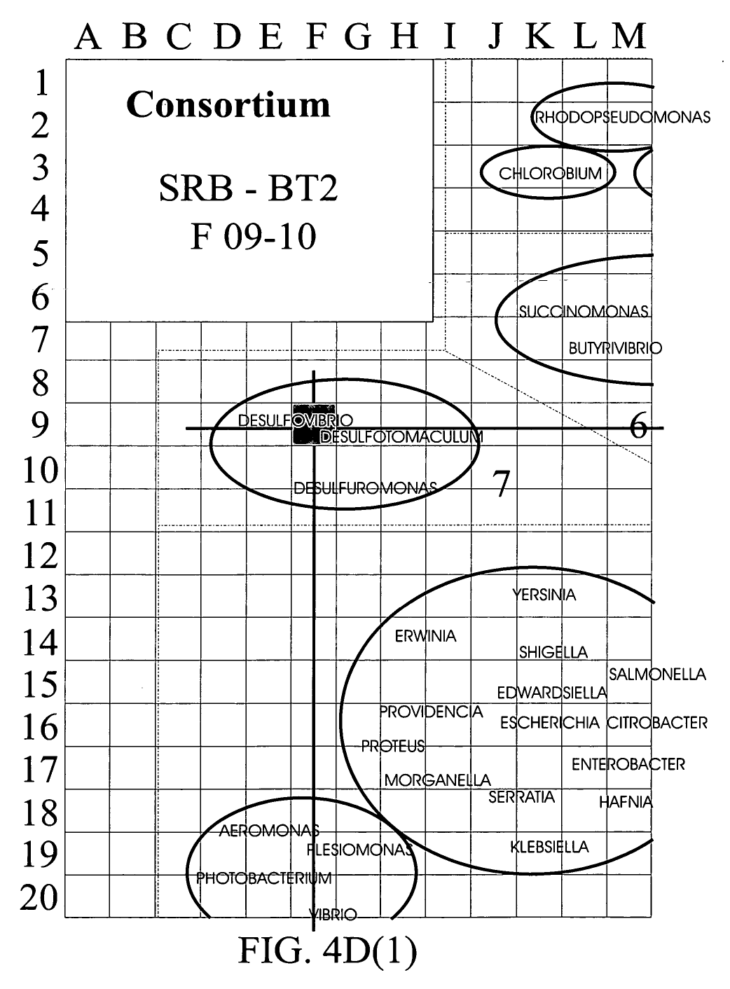

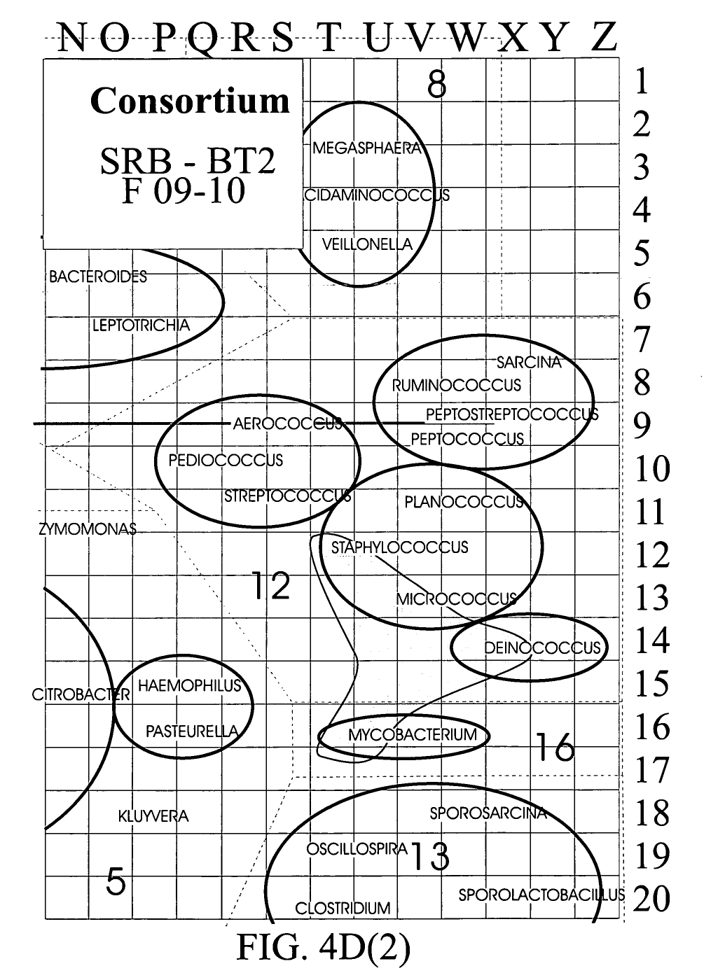

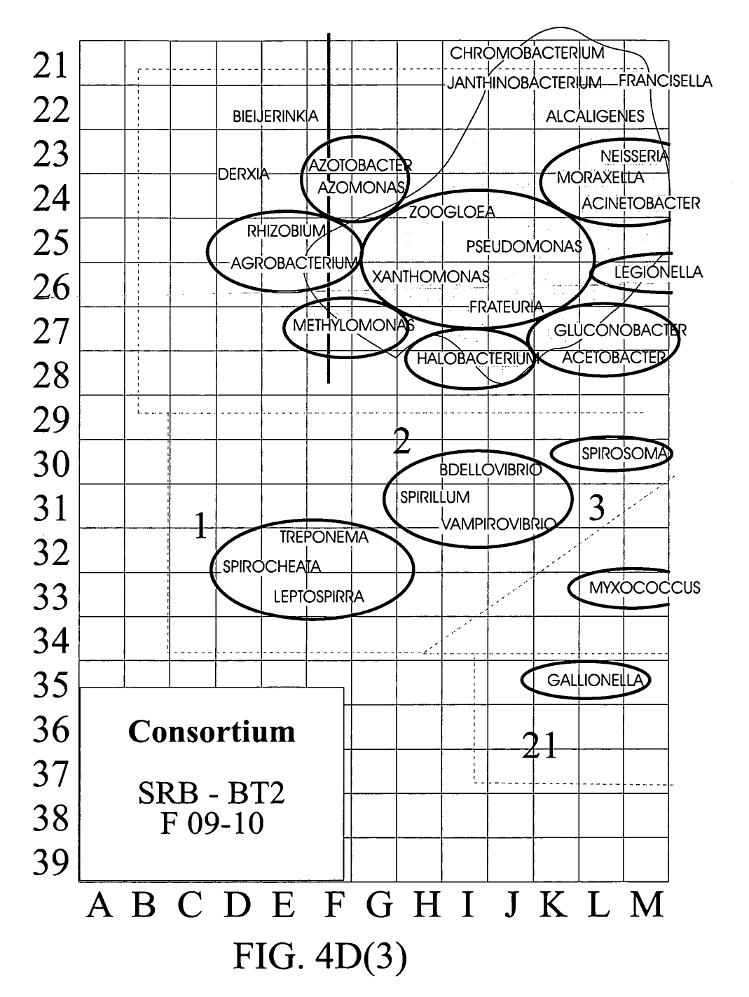

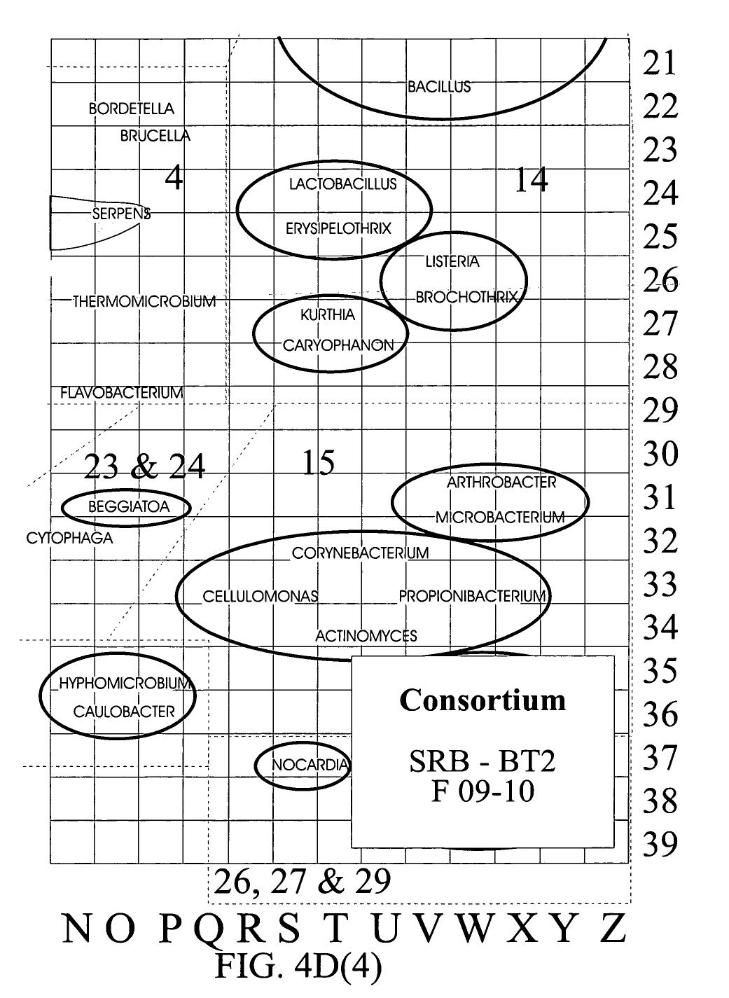

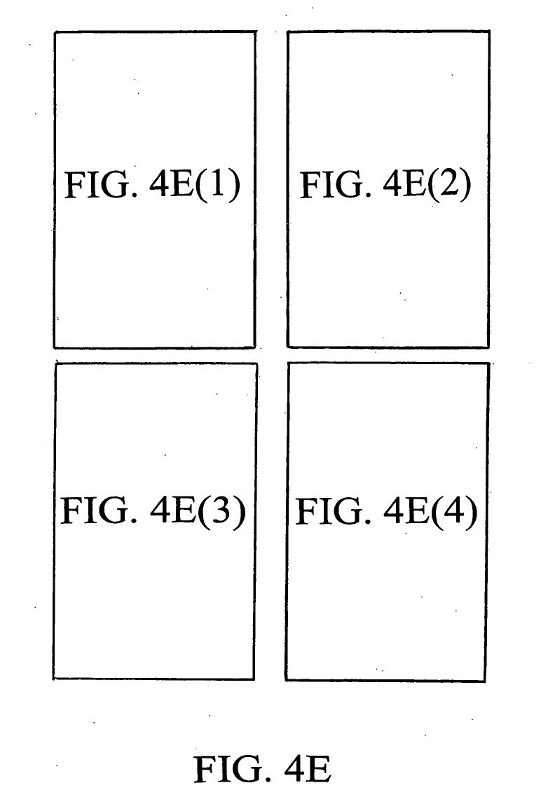

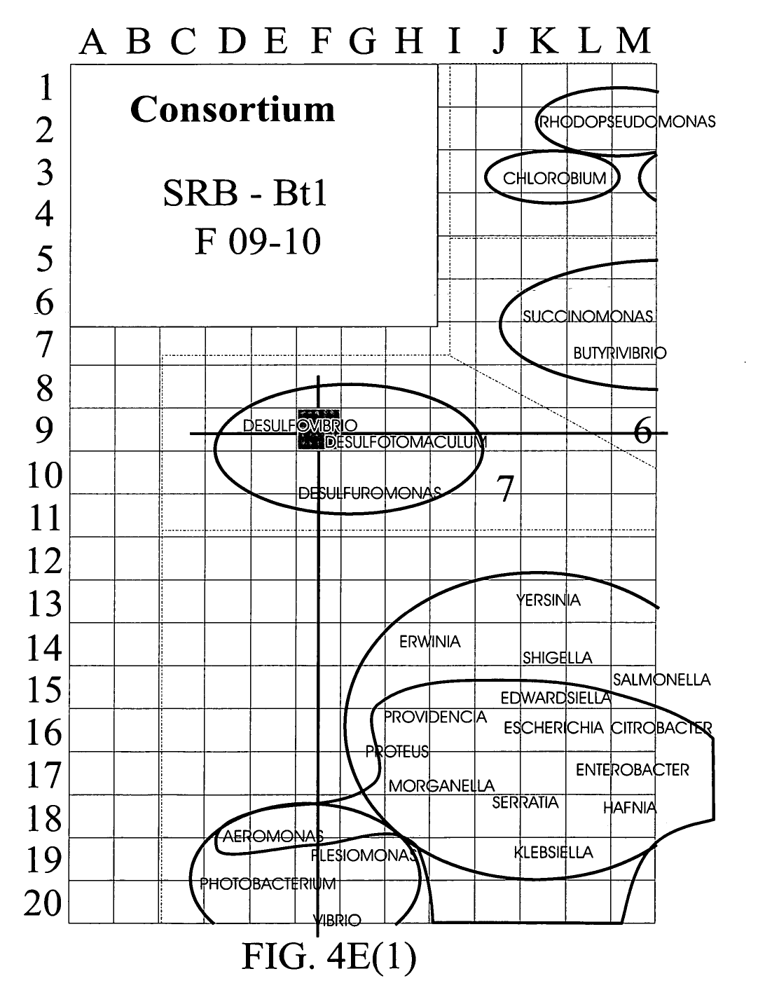

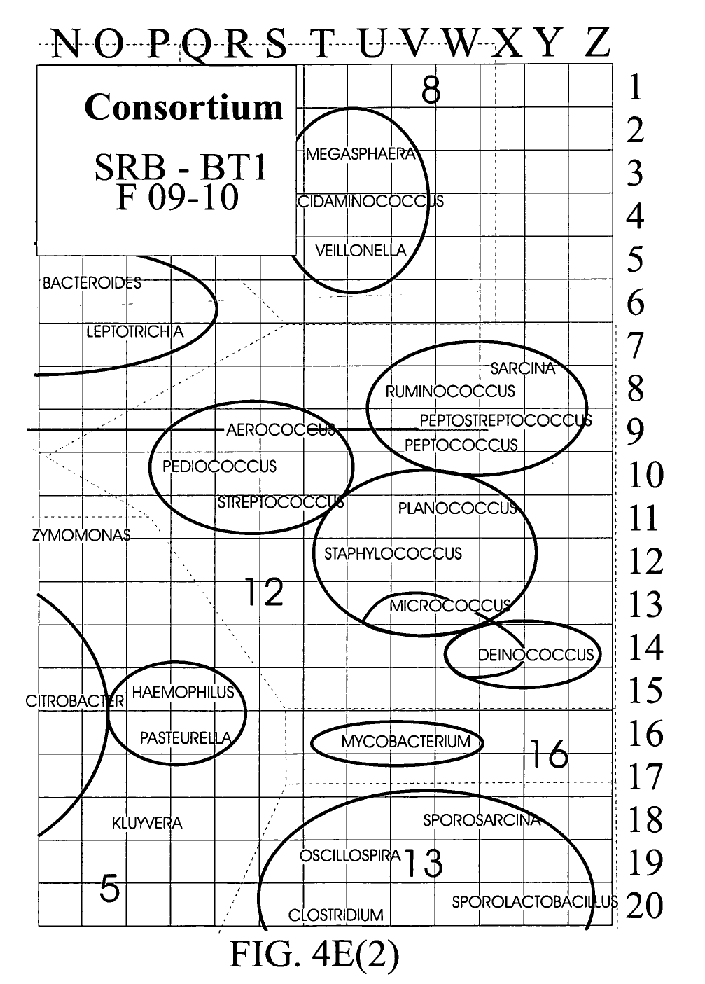

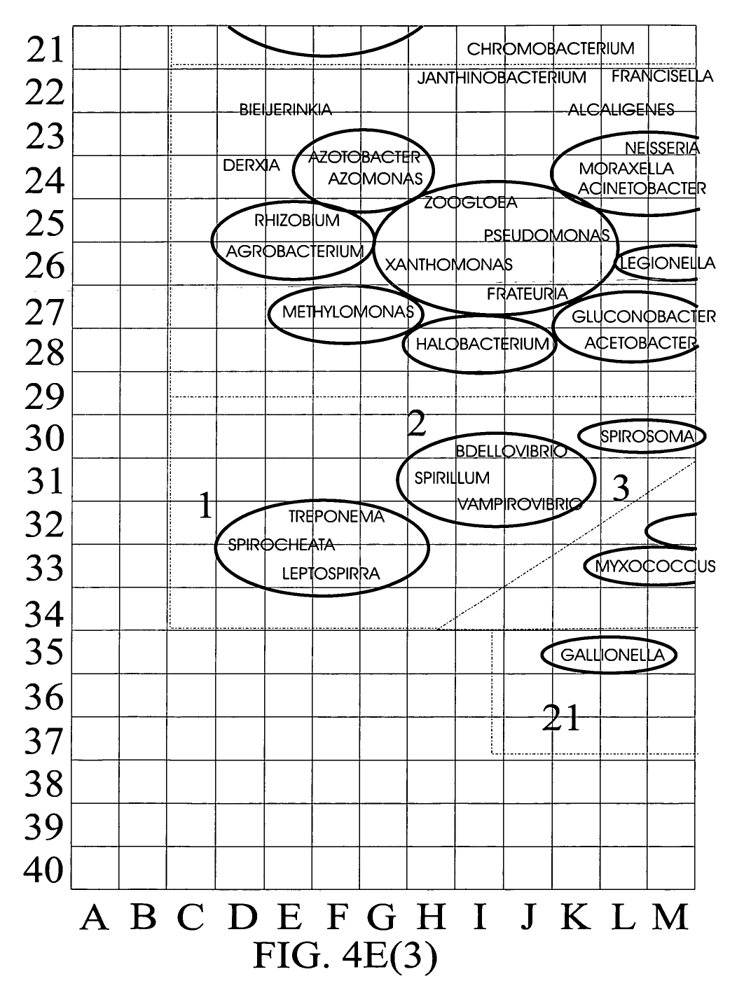

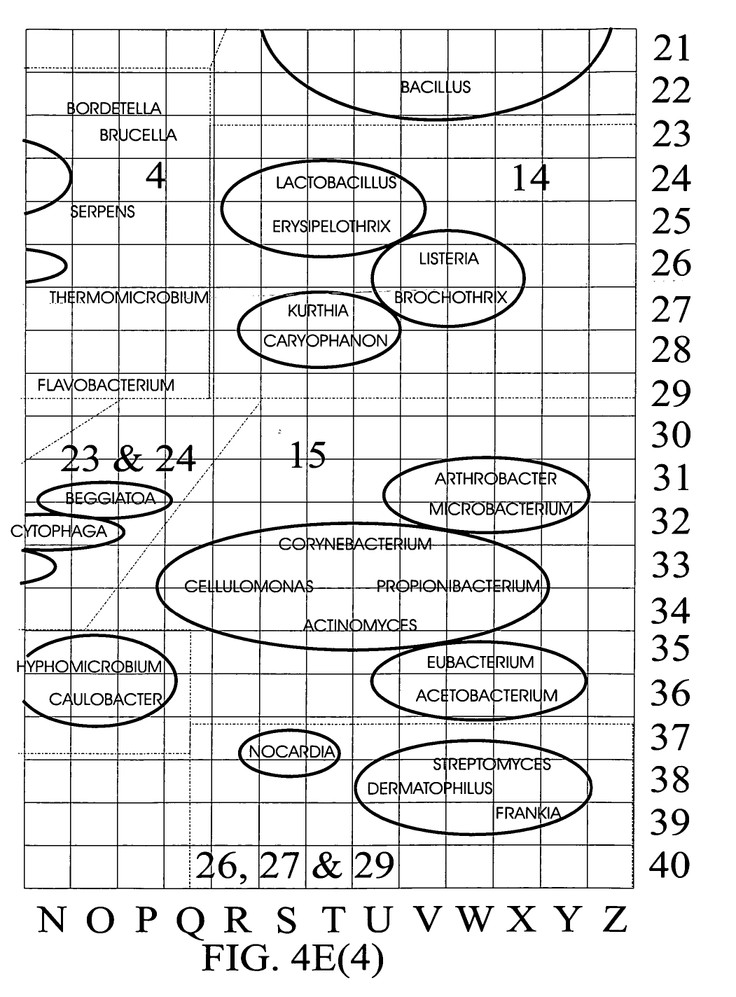

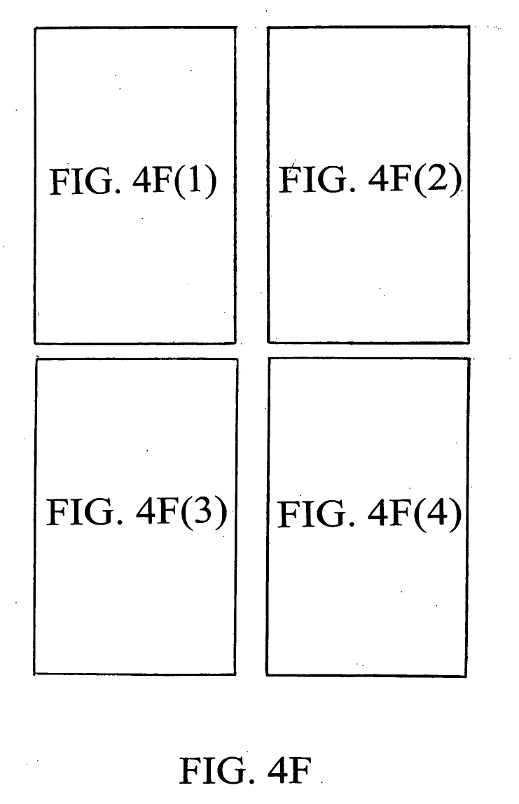

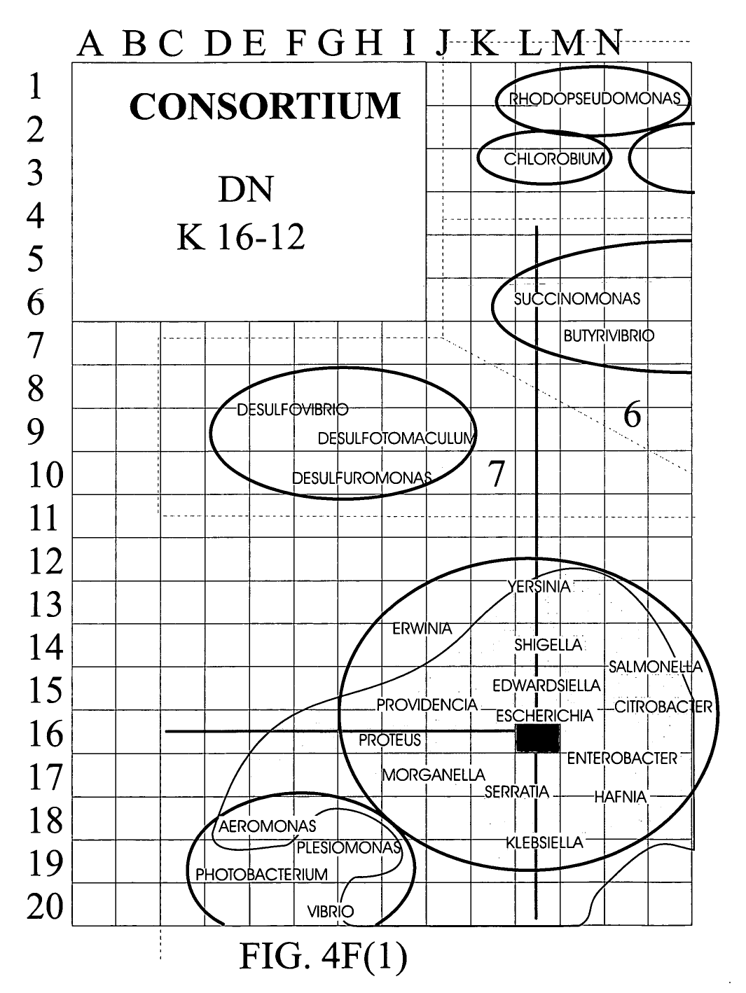

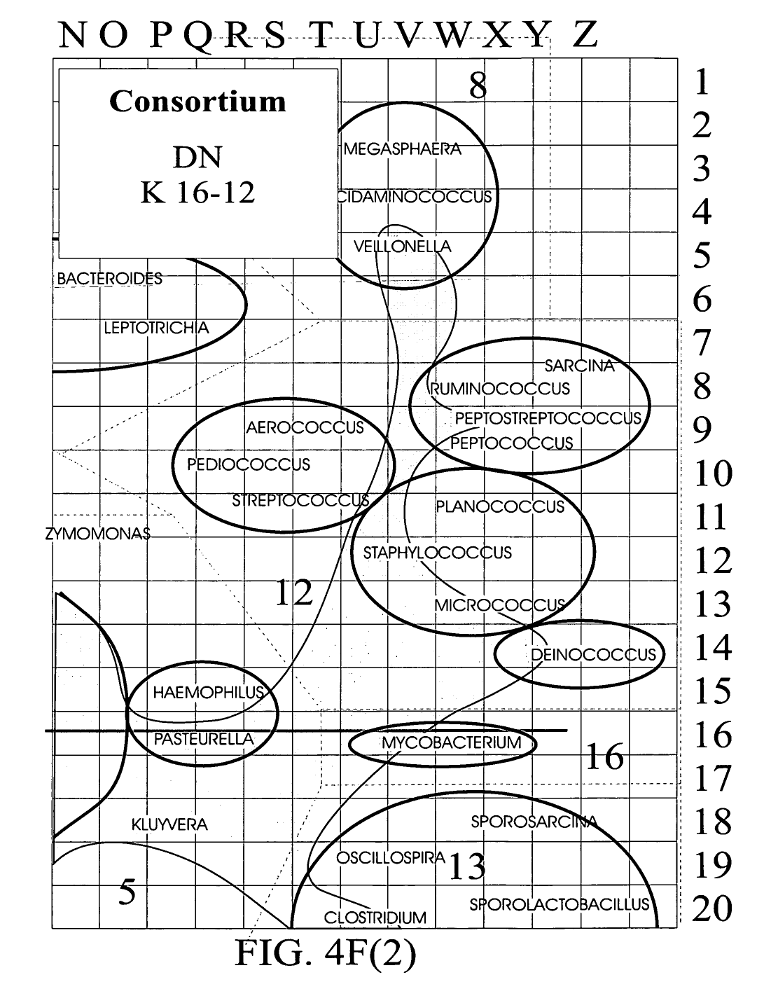

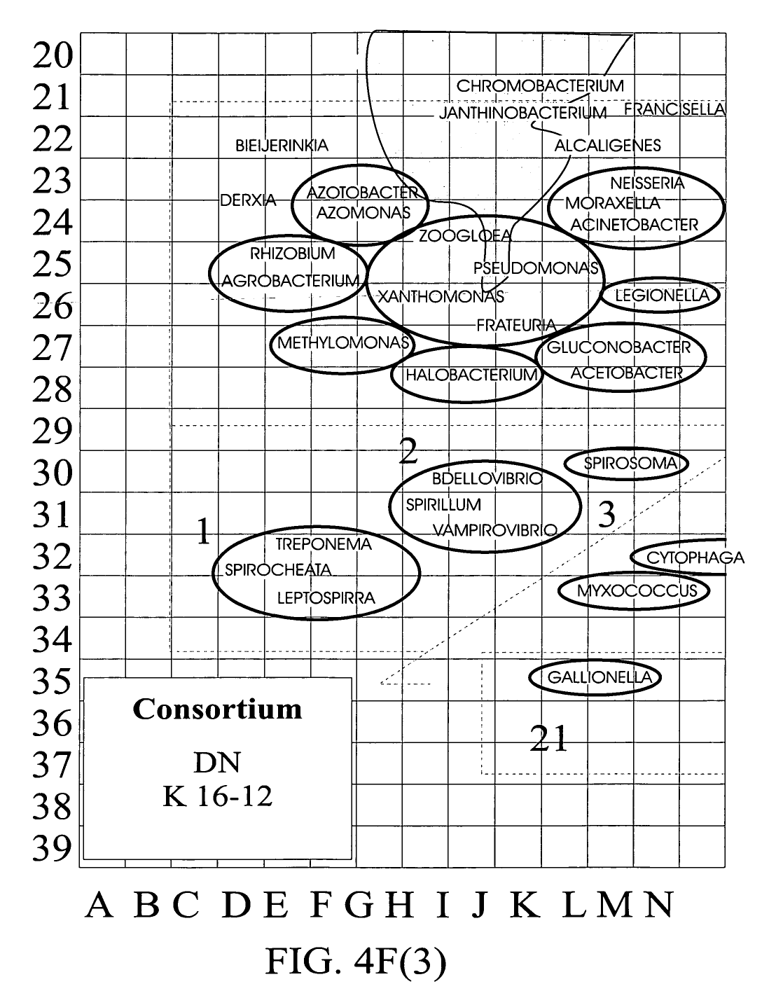

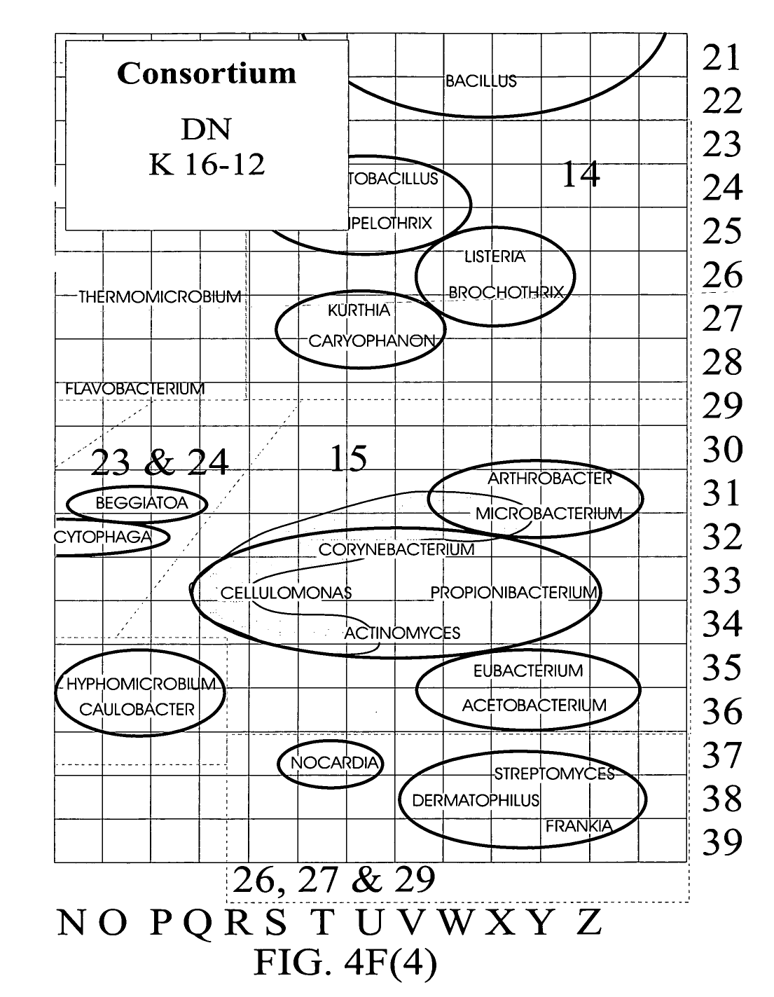

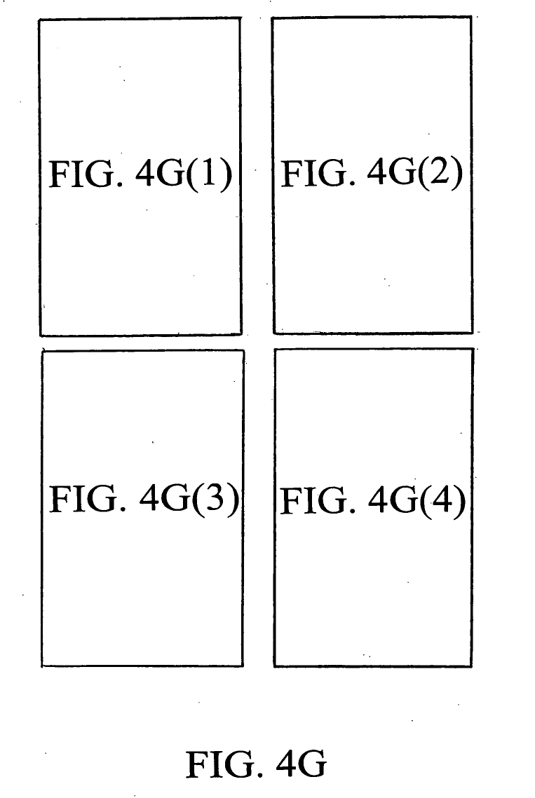

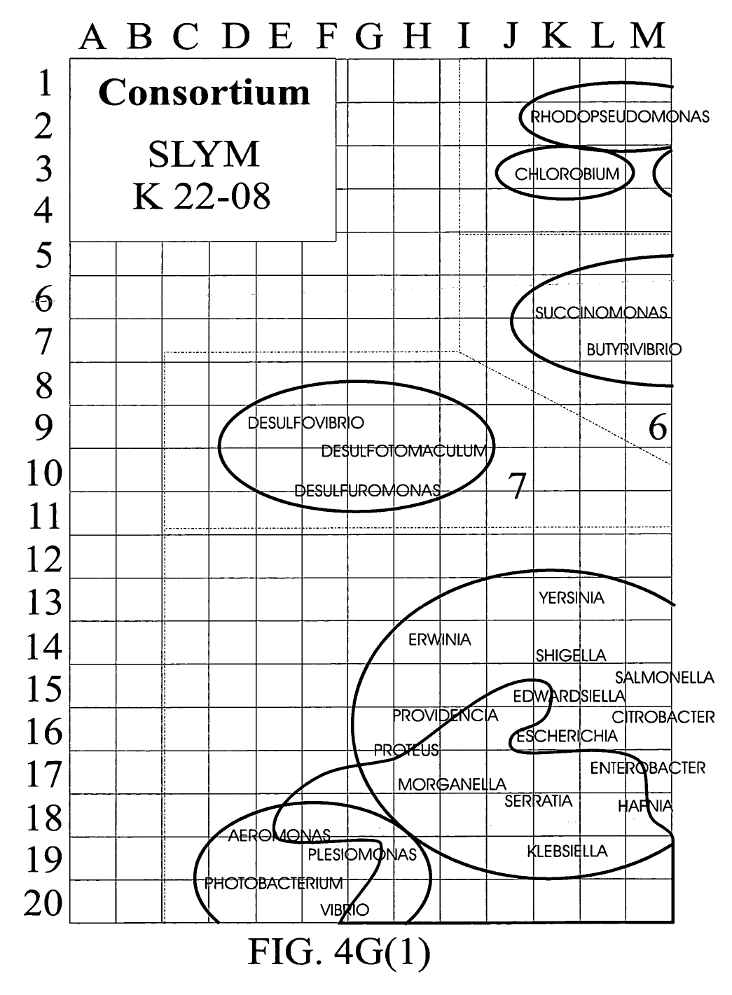

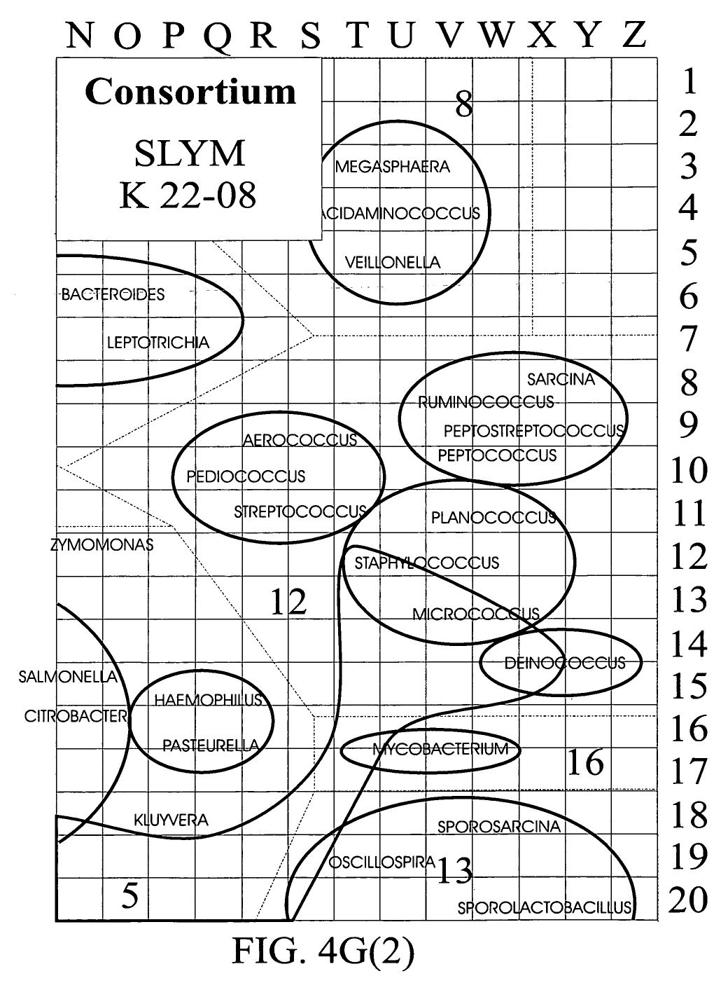

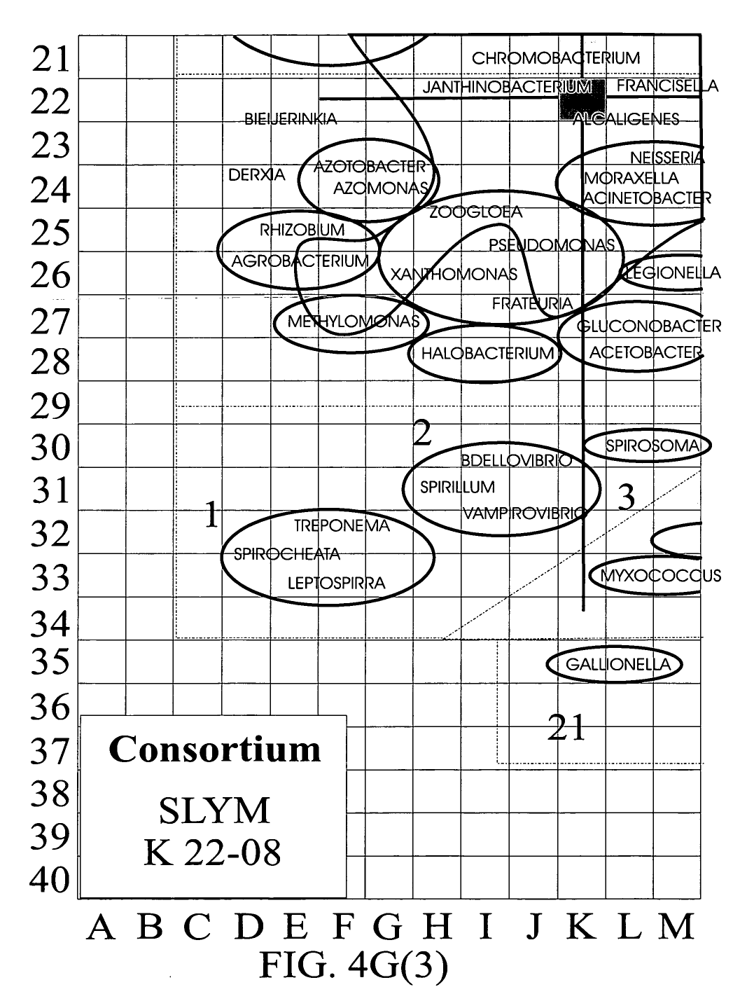

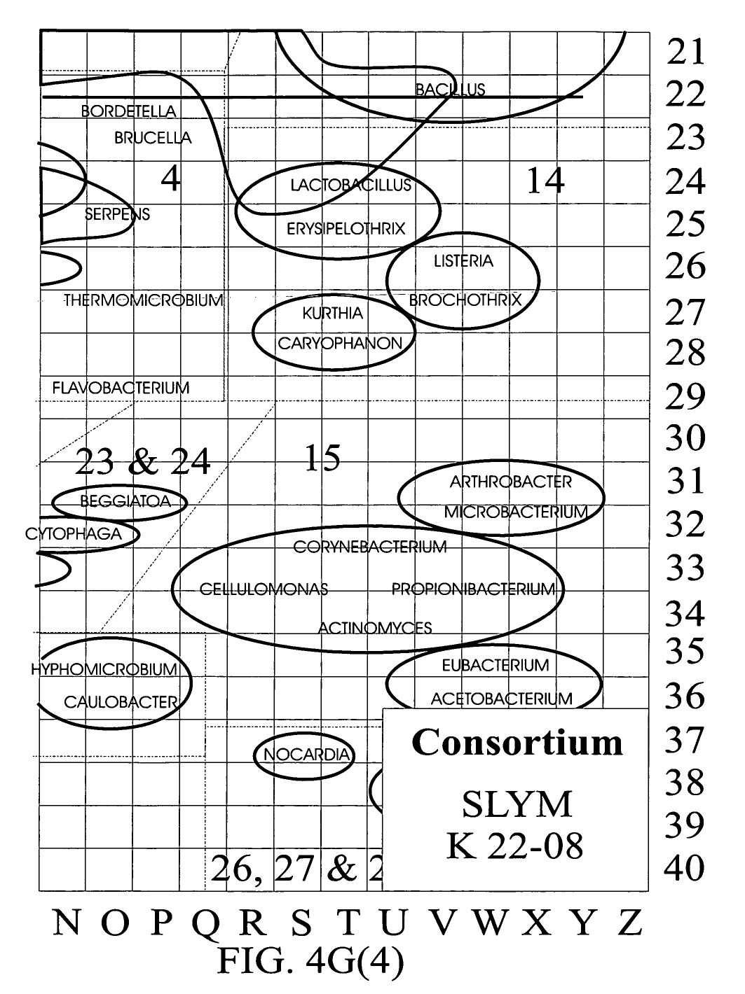

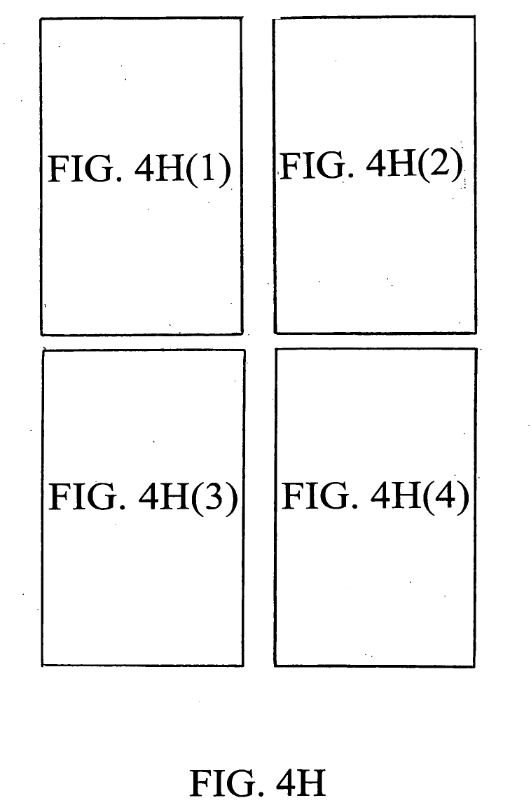

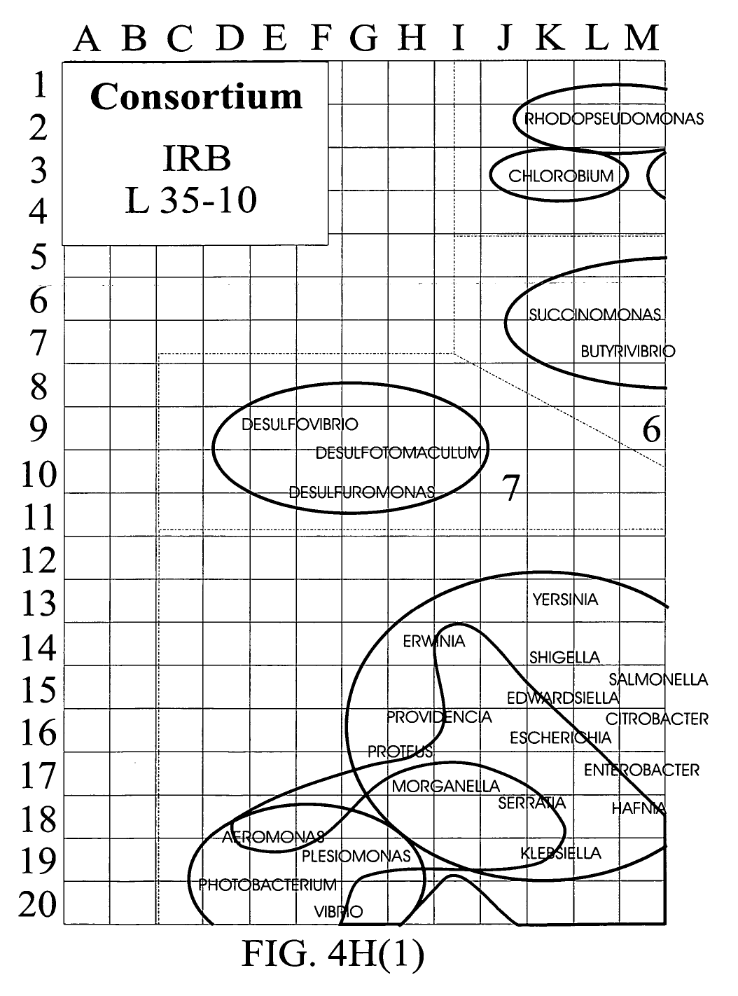

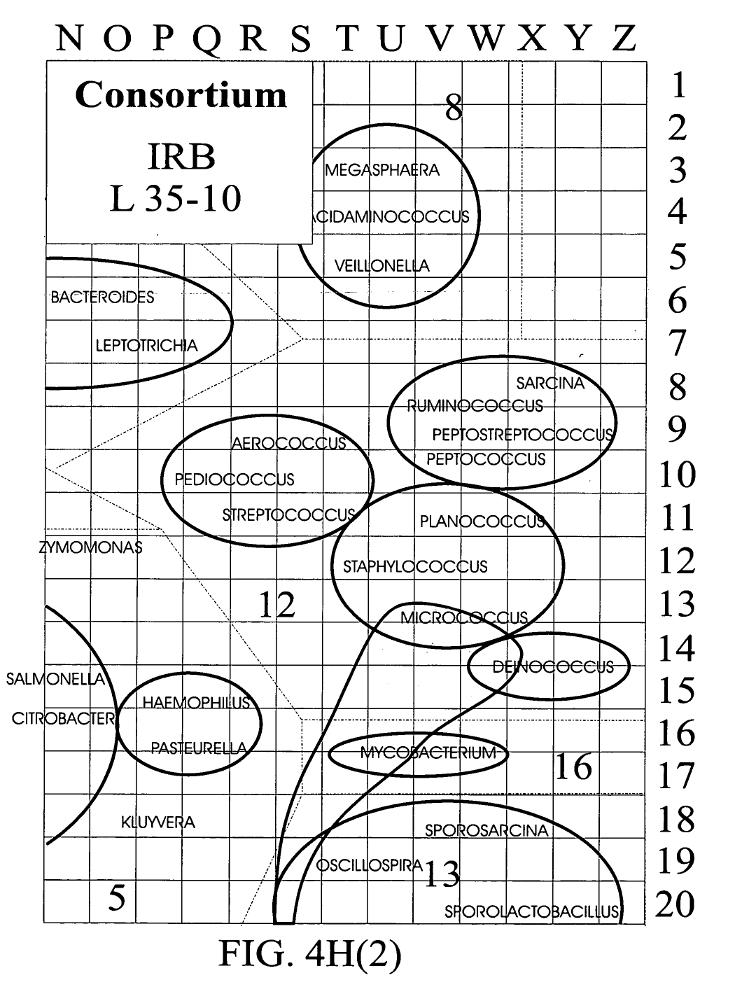

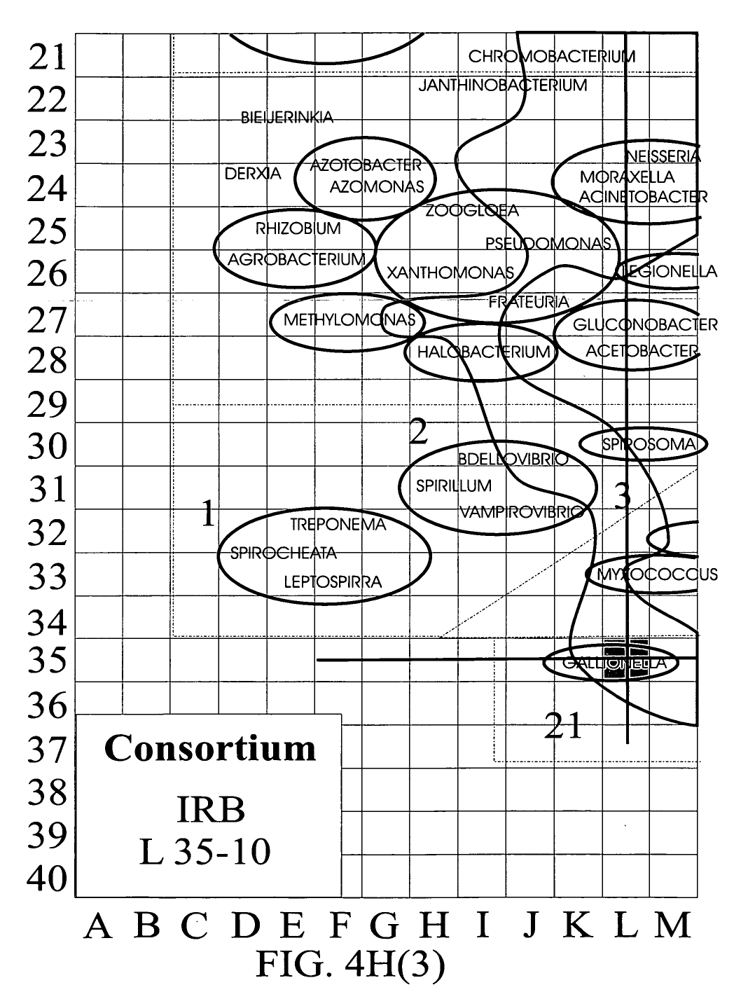

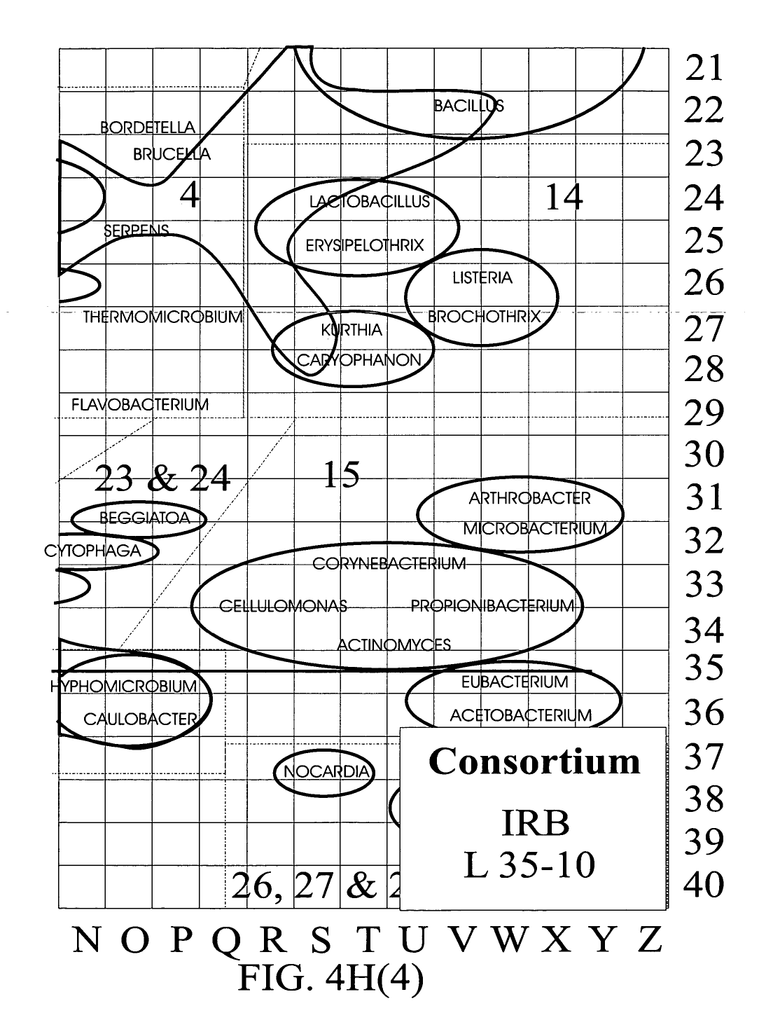

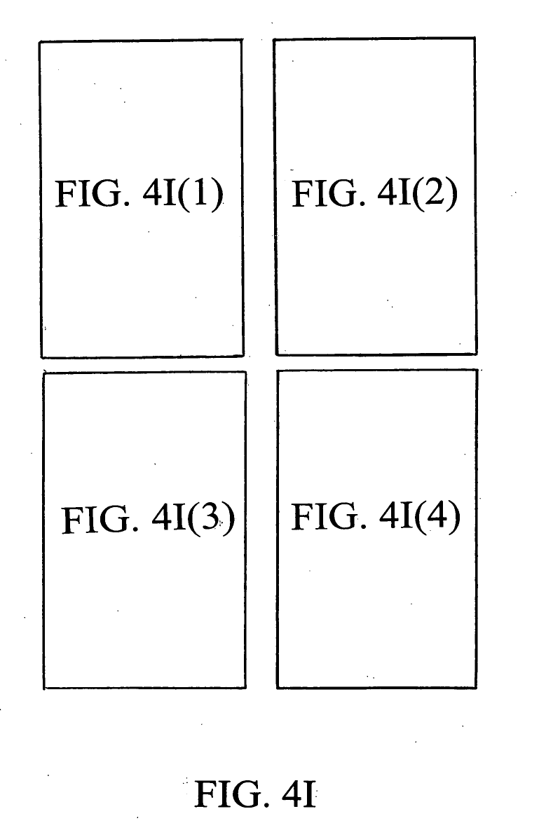

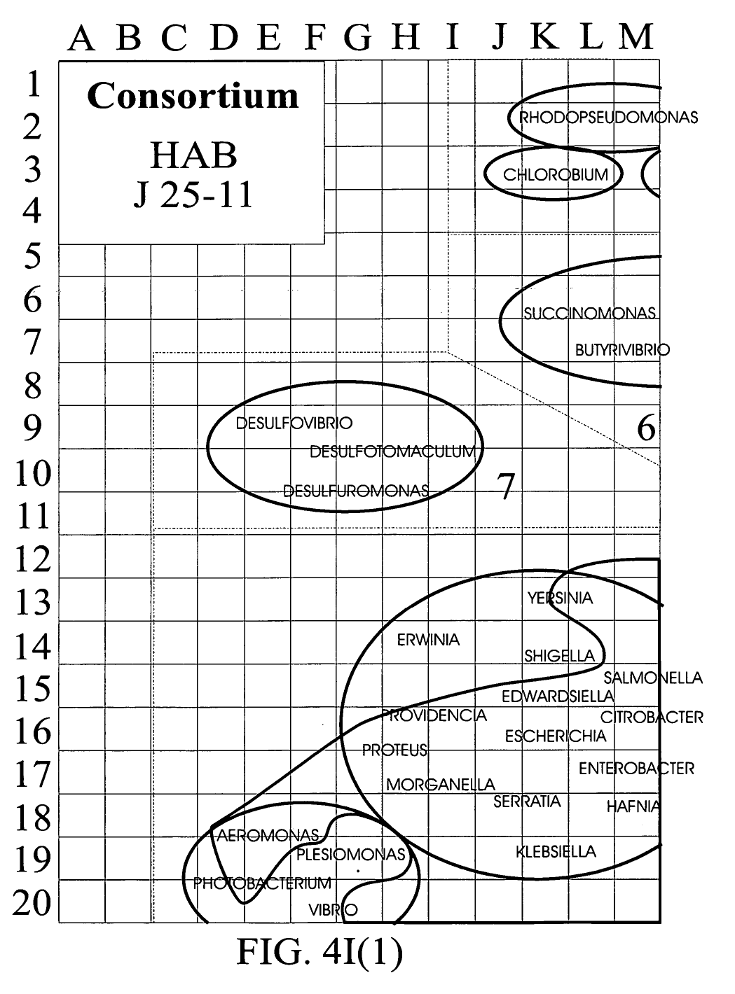

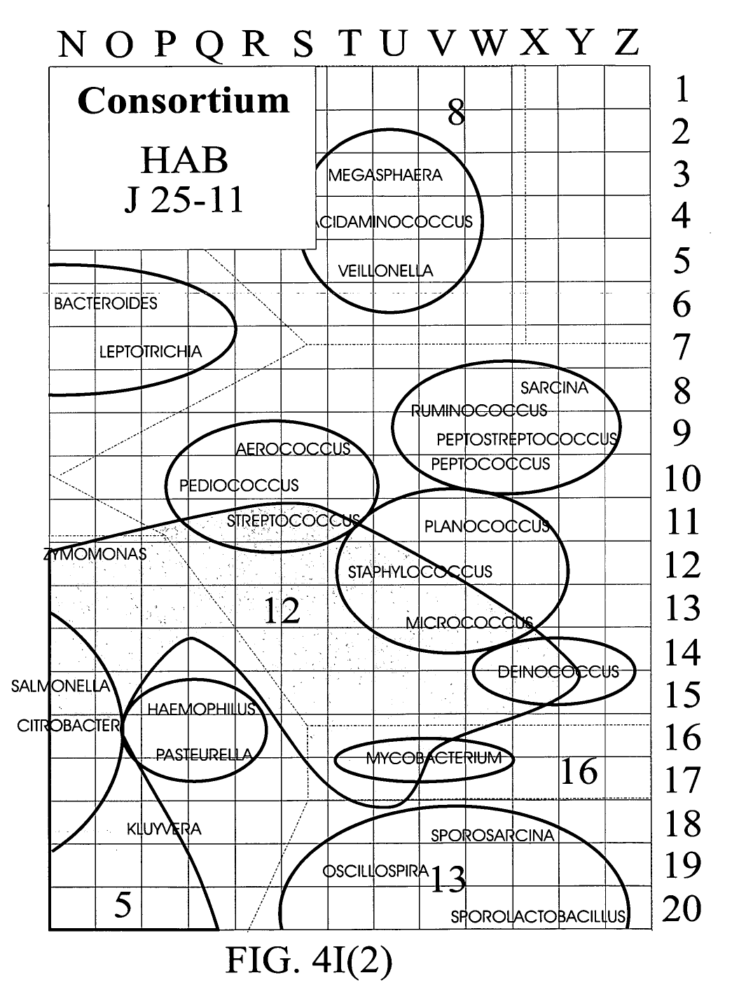

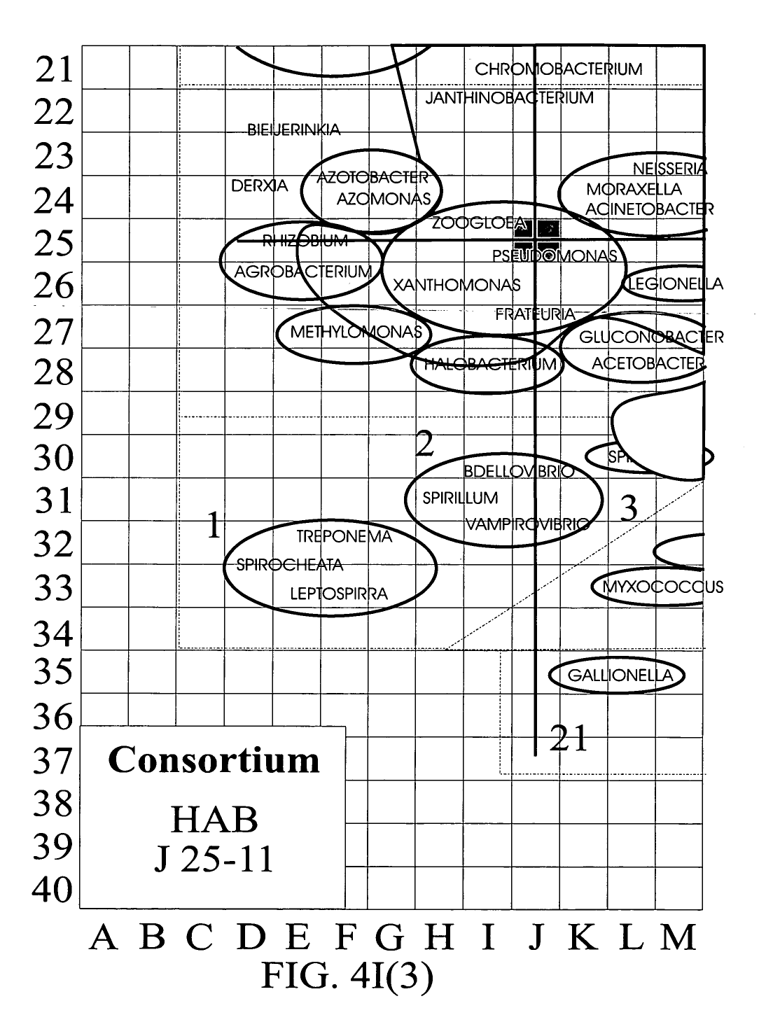

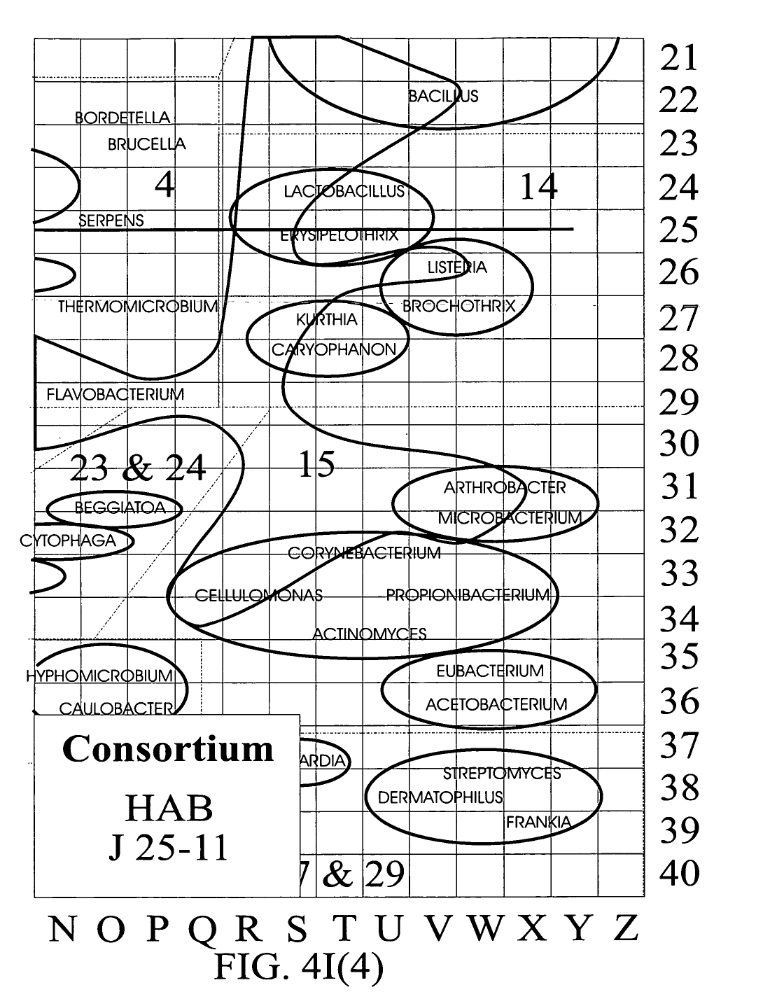

In order to identify the nature of consortia found in the rusticles, one of the present inventors has generated a microbial consortial identification code (MCIC) comprised of a two-dimensional atlas in which many of the major bacterial genera are displayed. Each genus is located within the atlas in a manner that reflects the relationship of that genus to neighboring genera. The development of the atlas makes it possible to define by-shade or cross-hatching the characteristics of individual genera. In the following description, the foregoing concept is extended to allow the generation of a method of coded MCIC by developing a grid reference system using the atlas, as shown in FIG. 4A, as the basis. By placing a referable grid structure onto the atlas, it now becomes possible to provide two important components to the generation of an MCIC. These are:

1. Focal grid site that would display the location of the heart of the consortium; and

2. Extension numeric reference that would indicate the dimensions of the consortium within the atlas.

To apply this approach to the identification of microbial consortia, the atlas has been placed within a grid having 26 columns and 39 rows for the bacterial genera. The columns are listed alphabetically from A to Z going from the left to the right and the rows are listed numerically from 1 to 39 in descending order. By applying this grid to the atlas, the location of individual genera can be determined by the focal grid site (FGS) created by the column (alphabetic) and row (numeric). For example, Pseudomonas would be located as J25, Escherichia as K16 and Bacillus as V21. These FGS locations are represented as single shaded boxes within the grid where the genus is considered to originate. Although subdivision of each grid box could allow the location of individual species within a genus, the concept herein is to create locational reference points for bacterial consortia centered at an FGS location considered to be the most appropriate for the consortium being defined.

In the definition of a specific consortium, the location of individual genera within the atlas can be achieved by applying shading of the grid areas within which genera harmonizing within the consortium are found. This shaded area for the consortial grid locations (CGL) would be irregular in form but would also embrace those species within the individual genera that are not active within the consortium being defined. The CGL would therefore set the outer limits of the consortium through focusing on the participating genera rather than species. The extent of the consortium spread over the grid is shown as the CGL but quantified by the mean distance that the extension moves away from a designated FGS for the consortium. Distance of consortial penetration across the atlas grid is referred to as the consortial penetration number (CPN, in two digits). This is defined as the mean number of grid boxes within the grids going laterally or vertically to the maximum extent in all four of the primary directions from the FGS defining the consortium. This mean distance expressed as the CPN would reflect the degree to which the genera within said defined consortium extend across the atlas grid. Each of the four lateral and vertical extensions from the FGS is defined as thin continuous lines that track from the FGS to the outermost edge defined for the consortium as being shaded. These lines are referred to as defining lines for the consortium (DFC) each of which has a specific number of grid boxes assigned from the FGS to the outermost edge of the consortial shade zone in that direction.

Three primary components are therefore involved in the definition of a consortium. These are defined as: (1) FGS location on the grid; (2) the numbers of grids involved in the extension of the DFC in all four quadrant directions from the FGS; and (3) the consortial position number (CPN) generated as the mean value for the four quadrant extensions of the DFC measured in whole grid boxes away from the FGS.

An MCIC is a reflection of the location of the genera which each collectively a part of the components that creates a consortium of interacting genera. Using the concepts and grid location system defined above, an MCIC designating a specific consortium would include the following: (1) FGS location, (2) followed by a dash, and then (3) followed by a two digit CPN. For example a consortium having an MCIC of J25-03 would be dominated by Pseudomonas with other members of the consortium closely related to that genus likely, in the same family, and neighboring families. If the MCIC was J25-12 then this would still indicate that Pseudomonas was a major member of the consortium but that other members of the consortium coming from the designated (shaded) area of 144 grid boxes out of the composite of 1,014 grid boxes in the bacterial atlas.

Reference would have to be made to the master atlas sheet specifically for the designated consortium to determine which genera that were actually a part of said consortium. This atlas sheet would include the focal grid location in a dark shade followed by a dash and the CPN in two digits. An initial list of the first designated MCIC for different consortia found in rusticles are listed below in FIG. 4B and the grids for some of these consortia are shown in FIGS. 4C-4I.

Provisional MCIC are also being applied to Archaea bacteria, algae and the Fungi as extensions to the atlas. Provisionally the Archaea-bacteria will occupy grids positions within columns A to G and from rows 29 to 39. These are provisionally designated under the MCIC of D35-05. Fungi will be classified within the extension of rows 40 to 79 and are provisionally listed as N60-20. Cyanobacteria and the algae occupy a grid above the bacteria atlas using the same column designation but with the rows listed in an ascending order alphabetically. Cyanobacteria are provisionally designated with the MCIC of NC-05 and the green algae have a provisional designation of NG-10. For all of these groups discussed directly above, the MCIC will be clarified as the atlas is built to cover the microorganisms other than the bacteria.

We claim:

1. A method of producing a mass culture of a rusticle consortia, comprising the steps of:
providing a growth substrate;
placing one or more samples obtained from a rusticle on the growth substrate;
placing the growth substrate with the samples thereon into a tank filled with an aqueous solution; and
controlling an environment of the aqueous solution to facilitate growth of the rusticle on the growth substrate.

2. A method according to claim 1; wherein the growth substrate comprises an anodically charged metal.

3. A method according to claim 2; wherein the growth substrate comprises a mild steel plate.

4. A method according to claim 3; further comprising the step of embrittling the mild steel plate.

5. A method according to claim 4; wherein the step of embrittling comprises hammering the steel plate at about 3000 p.s.i. for approximately 60 minutes.

6. A method according to claim 3; wherein the mild steel plate has a chemical composition comprising at least 0.8% phosphorus, at least 1.2% sulfur, and at least 1.4% manganese.

7. A method according to claim 3; further comprising the step of rolling the mild steel plate to promote the formation of lateral pearlite deposits within the steel plate to cause a flaking form of corrosion.

8. A method according to claim 1; wherein the growth substrate is formed of unlike metals.

9. A method according to claim 8; wherein the unlike metals are Fe and Cu.

10. A method according to claim 1; wherein the aqueous solution comprises water containing at least five ppm of oxygen and a nutrient substrate containing at least 1% of ferric ammonium citrate and inorganic nutrients.

11. A method according to claim 10; wherein the step of controlling an environment of the aqueous solution comprises maintaining a pH level of the aqueous solution within the range of 7.4 and 8.4.

12. A method according to claim 11; wherein the step of controlling an environment of the aqueous solution further comprises the step of maintaining a salt concentration of the aqueous solution in a range of 0.3 to 0.8%.

13. A method according to claim 12; wherein the step of controlling an environment of the aqueous solution further comprises the step of controlling a concentration of phosphorus to be within the range of 0.05 and 0.15 ppm.

14. A method according to claim 13; wherein the step of controlling an environment of the aqueous solution further comprises the step of controlling a temperature of the culture in an ambient room temperature range.

15. A method according to claim 1; further comprising the step of applying a charge to a location near the growth substrate to form a concentration of particles given off by the rusticle.

16. A therapeutic substance comprising a mass culture of a rusticle consortia produced according to claim 1.

17. A therapeutic substance comprising a substance produced by a mass culture of a rusticle consortia produced according to claim 1.

18. An antimicrobial substance comprising a mass culture of a rusticle consortia produced according to claim 1.

19. An antibiotic substance comprising a mass culture of a rusticle consortia produced according to claim 1.
